(19) United States
(12) Patent Application Publication
(10) Pub. No.: US 2014/0323824 A1
(43) Pub. Date: Oct. 30, 2014

(54) SYSTEMS AND METHODS FOR DETERMINING FLUID RESPONSIVENESS

(71) Applicant: Covidien LP, Mansfield, MA (US)

(72) Inventors: Paul S. Addison, Edinburgh (GB); Rui Wang, Livingston (GB); James Watson, Dunfermline (GB)

(73) Assignee: Covidien LP, Mansfield, MA (US)

(21) Appl. No.: 14/262,603

(22) Filed: Apr. 25, 2014

Related U.S. Application Data
(60) Provisional application No. 61/815,804, filed on Apr. 25, 2013.
(60) Provisional application No. 61/815,886, filed on Apr. 25, 2013.

(51) Int. Cl. A61B 5/00; A61B 5/08; A61B 5/1455

(52) U.S. Cl. CPC A61B 5/0082; A61B 5/14552; A61B 5/08
USPC 600/301; 600/473

(57) ABSTRACT

Provided are systems and methods for processing a physiological signal in order to determine fluid responsiveness of a subject. In some embodiments, a respiration rate of the subject is received or determined, the signal is filtered based on the respiration rate to generate a filtered signal, and the filtered signal is processed to determine fluid responsiveness. In some embodiments, regular respiration is detected and fluid responsiveness is determined when regular respiration is detected. In some embodiments, the respiration of a subject is controlled, and fluid responsiveness is determined during controlled respiration.

### CROSS REFERENCE TO RELATED APPLICATIONS

This application claims the benefit of U.S. Provisional Application No. 61/815,804, filed Apr. 25, 2013, and U.S. Provisional Application No. 61/815,886, filed Apr. 25, 2013, both of which are hereby incorporated by reference herein in their entireties.

### SUMMARY

The present disclosure relates to physiological signal processing, and more particularly relates to determining fluid responsiveness in a subject.

Methods and systems are provided for determining fluid responsiveness of a subject. In some embodiments a physiological signal is received by a system. In some embodiments, a respiration rate of the subject is received or determined, the signal is filtered based on the respiration rate to generate a filtered signal, and the filtered signal is processed to determine fluid responsiveness. In some embodiments, regular respiration is detected and fluid responsiveness is determined when regular respiration is detected. In some embodiments, the respiration of a subject is controlled, and fluid responsiveness is determined during controlled respiration.

The present disclosure provides embodiments for a system comprising a signal input, a respiration rate module, a filter module, a fluid responsiveness module, and an output module. The signal input is configured to receive a plethysmograph signal. The respiration rate module is coupled to the input and configured to calculate a respiration rate of a subject based at least in part on the plethysmograph signal. The filter module is coupled to the input and to the respiration rate module, and is configured to filter the plethysmograph signal based at least in part on the respiration rate to generate a filtered signal. The fluid responsiveness module is coupled to the filter module and configured to process the filtered signal to determine a value indicative of fluid responsiveness of the subject. The output module configured to provide an indication of the fluid responsiveness of the subject based at least in part on the value indicative of fluid responsiveness.

The present disclosure provides embodiments for a system comprising a respiration rate input, a signal input, a band-pass filter, a fluid responsiveness module, and an output module. The respiration rate input receives a respiration rate value for a subject. The signal input receives a plethysmograph signal. The band-pass filter is coupled to the respiration rate input and to the signal input, and is configured to filter the plethysmograph signal to generate a filtered signal. At least one characteristic of the band-pass filter is set based at least in part on the respiration rate. The fluid responsiveness module is coupled to the band-pass filter and is configured to process the filtered signal to determine a value indicative of fluid responsiveness of the subject. The output module is configured to provide an indication of the fluid responsiveness of the subject based at least in part on the value indicative of fluid responsiveness.

The present disclosure provides embodiments for a method comprising receiving at a signal input a plethysmograph signal from a sensor attached to a subject. The method further comprises filtering, using a filter module, the plethysmograph signal based on a respiration rate of the subject and processing the filtered signal using a processor to determine a value indicative of fluid responsiveness of the subject. The method further comprises outputting on an output device an indication of the fluid responsiveness of the subject based at least in part on the value indicative of fluid responsiveness.

The present disclosure provides embodiments for a system comprising a signal input, a respiration control input, a fluid responsiveness module, and an output module. The signal input receives a plethysmograph signal. The respiration control input receives information from a respiration control module that is capable of controlling breathing in a subject. The fluid responsiveness module is coupled to the signal input and the respiration control input and is configured to process the plethysmograph signal to determine a value indicative of fluid responsiveness of the subject. The output module is configured to provide an indication of the fluid responsiveness of the subject based at least in part on the value indicative of fluid responsiveness of the subject when the breathing in the subject is sufficiently controlled by the respiration control module.

The present disclosure provides embodiments for a system comprising a signal input, a ventilator input, a respiration detection module, a fluid responsiveness module, and an output module. The signal input receives a plethysmograph signal. The ventilator input is configured to receive information from an adjustable ventilator that is capable of providing varying degrees of control of breathing of a subject. The respiration detection module is coupled to the signal input and to the ventilator input and is configured to detect regular and irregular breathing in the subject based on at least one of the plethysmograph signal and the ventilator input. The fluid responsiveness module is coupled to the respiration detection module and is configured to process the plethysmograph signal to determine a value indicative of fluid responsiveness of the subject. The output module is configured to provide an indication of the fluid responsiveness of the subject based at least in part on the value indicative of fluid responsiveness during at least one of a time period of regular breathing detected by the respiration detection module and a time period of sufficiently controlled breathing by the adjustable ventilator.

The present disclosure provides embodiments for a system comprising a signal input, a respiration detection module, a fluid responsiveness module, and an output module. The signal input receives a plethysmograph signal. The respiration detection module is coupled to the signal input and is configured to detect whether regular breathing is present in a subject based on the plethysmograph signal. The fluid responsiveness module is coupled to the respiration detection module and is configured to process the plethysmograph signal to determine a value indicative of fluid responsiveness of the subject. The output module is configured to provide an indication of the fluid responsiveness of the subject based at least in part on the value indicative of fluid responsiveness when regular breathing is detected by the respiration detection module.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows a block diagram of an illustrative physiological monitoring system in accordance with some embodiments of the present disclosure;

FIG. 2A shows an illustrative plot of a light drive signal in accordance with some embodiments of the present disclosure;

FIG. 2B shows an illustrative plot of a detector signal that may be generated by a sensor in accordance with some embodiments of the present disclosure;

FIG. 3 is a perspective view of an illustrative physiological monitoring system in accordance with some embodiments of the present disclosure;

FIG. 4 shows an illustrative PPG signal that is modulated by respiration in accordance with some embodiments of the present disclosure;

FIG. 5 shows a comparison of portions of the illustrative PPG signal of FIG. 4 in accordance with some embodiments of the present disclosure;

FIG. 6 shows an illustrative PPG signal, a first derivative of the PPG signal, and a second derivative of the PPG signal in accordance with some embodiments of the present disclosure;

FIG. 7 shows an illustrative plot of a PPG waveform reflecting respiratory modulations used to determine fluid responsiveness in accordance with some embodiments of the present disclosure;

FIG. 8 shows illustrative steps for determining fluid responsiveness in accordance with some embodiments of the present disclosure;

FIG. 9 shows an illustrative physiological monitoring system or monitoring fluid responsiveness of a subject in accordance with some embodiments for the present disclosure;

FIG. 10 shows an illustrative plot of respiratory flow with periods of regular and irregular breathing in accordance with some embodiments of the present disclosure;

FIG. 11 shows illustrative steps for determining fluid responsiveness in accordance with some embodiments of the present disclosure; and

FIG. 12 shows an illustrative physiological monitoring system or monitoring fluid responsiveness of a subject in accordance with some embodiments for the present disclosure.

### DETAILED DESCRIPTION OF THE FIGURES

The present disclosure is directed towards determining fluid responsiveness in a subject. In particular, in some embodiments, systems and methods are configured to filter a physiological signal based on a respiration rate of the subject and determine fluid responsiveness of the subject based on the filtered signal. In some embodiments, systems and methods are configured to determine fluid responsiveness of the subject during regular breathing intervals. In some embodiments, systems and methods are configured to control the subject's breathing for a period of time and determine fluid responsiveness during the controlled periods.

Fluids are commonly delivered to a patient in order to improve the patient's hemodynamic status. Fluid is delivered with the expectation that it will increase the patient's cardiac preload, stroke volume, and cardiac output, resulting in improved oxygen delivery to the organs and tissue. Fluid delivery may also be referred to as volume expansion, fluid therapy, fluid challenge, or fluid loading. However, improved hemodynamic status is not always achieved by fluid loading. Moreover, inappropriate fluid loading may worsen a patient's status, such as by causing hypovolemia to persist (potentially leading to inadequate organ perfusion), or by causing hypervolemia (potentially leading to peripheral or pulmonary edema).

Respiratory variation in the arterial blood pressure waveform is known to be a good predictor of a patient's response to fluid loading, or fluid responsiveness. Fluid responsiveness represents a prediction of whether such fluid loading will improve blood flow within the patient. Fluid responsiveness refers to the response of stroke volume or cardiac output to fluid administration. A patient is said to be fluid responsive if fluid loading does accomplish improved blood flow, such as by an improvement in cardiac output or stroke volume index by about 15% or more. In particular, the pulse pressure variation (PPV) parameter from the arterial blood pressure waveform has been shown to be a good predictor of fluid responsiveness. This parameter can be monitored while adding fluid incrementally, until the PPV value indicates that the patient's fluid responsiveness has decreased, and more fluids will not be beneficial to the patient. This treatment can be accomplished without needing to calculate blood volume or cardiac output directly. This approach, providing incremental therapy until a desired target or endpoint is reached, may be referred to as goal-directed therapy (GDT).

However, determining the PPV is an invasive procedure, requiring the placement of an arterial line in order to obtain the arterial blood pressure waveform. This invasive procedure is time-consuming and presents a risk of infection to the patient. Respiratory variation in a photoplethysmograph (PPG) signal may provide a non-invasive alternative to PPV. The PPG signal can be obtained non-invasively, such as from a pulse oximeter. One measure of respiratory variation in the PPG is the Delta POP metric, which is a measure of the strength of respiratory-induced amplitude modulations of the PPG. This metric assesses changes in the pulse oximetry plethysmograph, and is abbreviated as ΔPOP or DPOP. In addition to DPOP, a number of other measures of respiratory variation may be used to determine fluid responsiveness, including other measures of respiratory-induced amplitude modulations, other respiratory-induced modulations, and any suitable combination thereof. While studies have shown a favorable correlation between DPOP and PPV, there exists a need for more accurate processing of signals to determine DPOP and other similar measures of fluid responsiveness.

One source of variability in determining fluid responsiveness lies in the underlying PPG signals (or other physiological signal) used to measure fluid responsiveness. PPG signals (and any other suitable physiological signal used to determine fluid responsiveness) often include noise and/or other unwanted components that may affect amplitude modulations or other relevant modulations. These other components may be due to a number of factors, including patient movement, ectopic heart beats and other arrhythmias, regions of physiological instability (e.g., dramatic changes in heart rate, vasotone, etc.), other similar factors, and a combination thereof. In order to isolate modulations primarily induced by respiration, it is thus desirable to remove, from the PPG or other physiological signal, all information not associated with respiratory-induced modulations.

In accordance with some embodiments of the present disclosure, a respiration rate may be determined based on the PPG signal or otherwise received from an external device. The respiration rate may be used to filter the PPG signal in order to remove non-respiratory-induced modulations in the signal. For example, the respiration rate may be used to set one or more frequency thresholds used to filter the PPG signal, and the fluid responsiveness may be determined based on the filtered signal.

Another source of variability in determining fluid responsiveness lies in the manner of the subject's breathing. Studies have shown that the correlation between DPOP and PPV is particularly strong when DPOP is determined during periods of controlled and/or regular breathing by the subject, as opposed to periods of sporadic breathing, where the correlation between DPOP and PPV can be degraded. As used herein, the term “regular breathing” may refer to a breathing pattern that exhibits minimal variation in any number of relevant characteristics, such as the pressure per breath, breath period, respiration rate, morphology of flow, morphology of pressure, any other suitable characteristic, or any suitable combination thereof. Conversely, the term “irregular breathing” may refer to a breathing pattern or absence thereof that exhibits excessive variation in any of the aforementioned characteristics. In accordance with some embodiments of the present disclosure, regular breathing in the subject may be detected, and fluid responsiveness may be determined based primarily on periods of regular breathing. As used herein, the term “controlled breathing” may refer to breathing that is assisted, induced, or otherwise influenced to minimize the variation of any of the aforementioned characteristics, whether by external device such as a ventilator, clinical treatment or instruction, or other suitable device or method for minimizing the variation of a subject's breathing. In accordance with some embodiments of the present disclosure, breathing of the subject may be controlled by a device, treatment, or other suitable method, and fluid responsiveness may be determined based primarily during controlled periods.

For purposes of clarity, the present disclosure is written in the context of the physiological signal being a PPG signal generated by a pulse oximetry system. It will be understood that any other suitable physiological signal or any other suitable system may be used in accordance with the teachings of the present disclosure.

The foregoing techniques may be implemented in an oximeter. An oximeter is a medical device that may determine the oxygen saturation of an analyzed tissue. One common type of oximeter is a pulse oximeter, which may non-invasively measure the oxygen saturation of a patient's blood (as opposed to measuring oxygen saturation invasively by analyzing a blood sample taken from the patient). Pulse oximeters may be included in patient monitoring systems that measure and display various blood flow characteristics including, but not limited to, the blood oxygen saturation (e.g., arterial, venous, or both). Such patient monitoring systems, in accordance with the present disclosure, may also measure and display additional or alternative physiological parameters such as pulse rate, respiration rate, respiration effort, blood pressure, hemoglobin concentration (e.g., oxygenated, deoxygenated, and/or total), systemic vascular resistance, mean arterial pressure, cardiac output, central venous pressure, oxygen demand, adaptive filter parameters, fluid responsiveness parameters, any other suitable physiological parameters, or any combination thereof.

Pulse oximetry may be implemented using a photoplethysmograph. Pulse oximeters and other photoplethysmograph devices may also be used to determine other physiological parameter and information as is known in the art.

An oximeter may include a light sensor that is placed at a site on a patient, typically a fingertip, toe, forehead or earlobe, or in the case of a neonate, across a foot or hand. The oximeter may use a light source to pass light through blood perfused tissue and photoelectrically sense the absorption of the light in the tissue. Additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, around or in front of the ear, and locations with strong pulsatile arterial flow. Suitable sensors for these locations may include sensors that detect reflected light.

The oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, an inverted signal, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some embodiments, the photonic signal interacting with the tissue is of one or more wavelengths that are attenuated by the blood in an amount representative of the blood constituent concentration. Red and infrared (IR) wavelengths may be used because it has been observed that highly oxygenated blood will absorb relatively less red light and more IR light than blood with a lower oxygen saturation. By comparing the intensities of two wavelengths at different points in the pulse cycle, it is possible to estimate the blood oxygen saturation of hemoglobin in arterial blood.

The system may process data to determine physiological parameters using techniques well known in the art. For example, the system may determine arterial blood oxygen saturation using two wavelengths of light and a ratio-of-ratios calculation. As another example, the system may determine regional blood oxygen saturation using two wavelengths of light and two detectors located at different distances from the emitters. The system also may identify pulses and determine pulse amplitude, respiration, blood pressure, other suitable parameters, or any combination thereof, using any suitable calculation techniques. In some embodiments, the system may use information from external sources (e.g., tabulated data, secondary sensor devices) to determine physiological parameters.

In some embodiments, a light drive modulation may be used. For example, a first light source may be turned on for a first drive pulse, followed by an off period, followed by a second light source for a second drive pulse, followed by an off period. The first and second drive pulses may be used to determine physiological parameters. The off periods may be used to detect ambient signal levels, reduce overlap of the light drive pulses, allow time for light sources to stabilize, allow time for detected light signals to stabilize or settle, reduce heating effects, reduce power consumption, for any other suitable reason, or any combination thereof.

It will be understood that the techniques described herein are not limited to pulse oximeters and may be applied to any suitable physiological monitoring device.

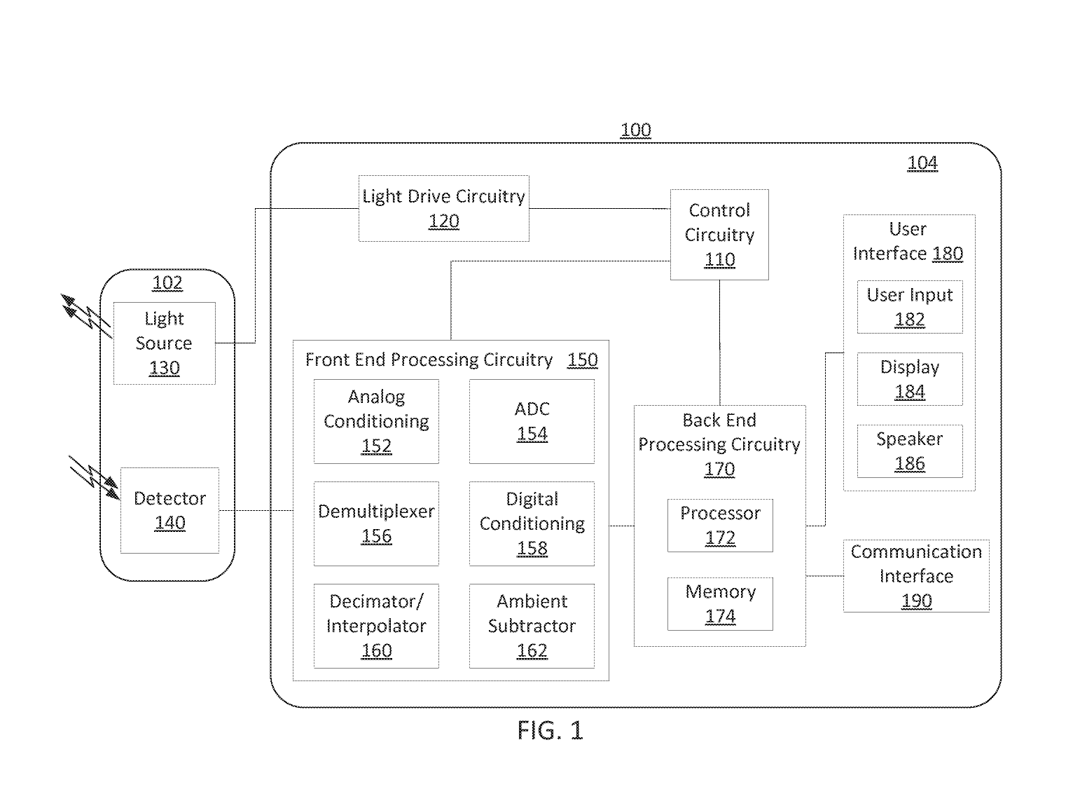

The following description and accompanying FIGS. 1-12 provide additional details and features of some embodiments of the present disclosure.

FIG. 1 shows a block diagram of illustrative physiological monitoring system 100 in accordance with some embodiments of the present disclosure. System 100 may include a sensor 102 and a monitor 104 for generating and processing sensor signals that include physiological information of a subject. In some embodiments, sensor 102 and monitor 104 may be part of an oximeter. In some embodiments, system 100 may include more than one sensor 102.

Sensor 102 of physiological monitoring system 100 may include light source 130 and detector 140. Light source 130 may be configured to emit photonic signals having one or more wavelengths of light (e.g. red and IR) into a subject's tissue. For example, light source 130 may include a red light emitting light source and an IR light emitting light source, e.g. red and IR light emitting diodes (LEDs), for emitting light into the tissue of a subject to generate sensor signals that include physiological information. In one embodiment, the red wavelength may be between about 600 nm and about 750 nm, and the IR wavelength may be between about 800 nm and about 1000 nm. It will be understood that light source 130 may include any number of light sources with any suitable characteristics. In embodiments where an array of sensors is used in place of single sensor 102, each sensor may be configured to emit a single wavelength. For example, a first sensor may emit only a red light while a second may emit only an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 140 may be chosen to be specifically sensitive to the chosen targeted energy spectrum of light source 130.

In some embodiments, detector 140 may be configured to detect the intensity of light at the red and IR wavelengths. In some embodiments, an array of sensors may be used and each sensor in the array may be configured to detect an intensity of a single wavelength. In operation, light may enter detector 140 after passing through the subject's tissue. Detector 140 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detector 140. After converting the received light to an electrical signal, detector 140 may send the detection signal to monitor 104, where the detection signal may be processed and physiological parameters may be determined (e.g., based on the absorption of the red and IR wavelengths in the subject's tissue). In some embodiments, the detection signal may be preprocessed by sensor 102 before being transmitted to monitor 104. Although only one detector 140 is depicted in FIG. 1, in some embodiments, sensor 102 may include additional detectors located at different distances from the light source 130. In embodiments with additional detectors, the sensitivity of the additional detectors may vary based on the distance between the detector and light source 130 such that a far detector may be more sensitive to light than a near detector.

In the embodiment shown, monitor 104 includes control circuitry 110, light drive circuitry 120, front end processing circuitry 150, back end processing circuitry 170, user interface 180, and communication interface 190. Monitor 104 may be communicatively coupled to sensor 102.

Control circuitry 110 may be coupled to light drive circuitry 120, front end processing circuitry 150, and back end processing circuitry 170, and may be configured to control the operation of these components. In some embodiments, control circuitry 110 may be configured to provide timing control signals to coordinate their operation. For example, light drive circuitry 120 may generate a light drive signal, which may be used to turn on and off the light source 130, based on the timing control signals. The front end processing circuitry 150 may use the timing control signals to operate synchronously with light drive circuitry 120. For example, front end processing circuitry 150 may synchronize the operation of an analog-to-digital converter and a demultiplexer with the light drive signal based on the timing control signals. In addition, the back end processing circuitry 170 may use the timing control signals to coordinate its operation with front end processing circuitry 150.

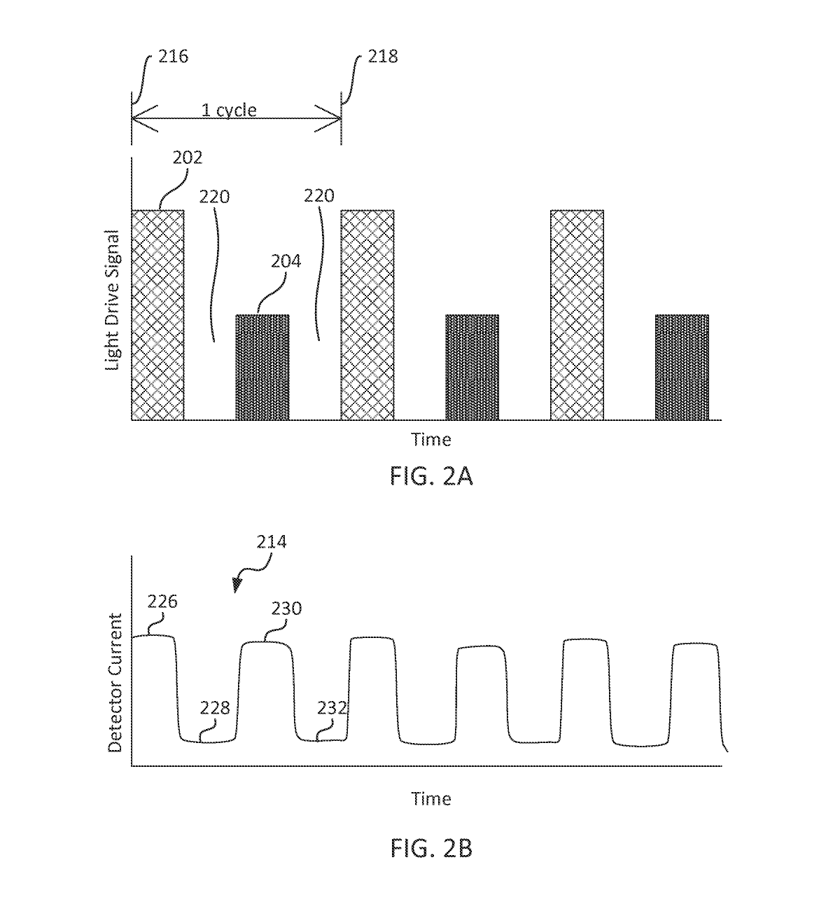

Light drive circuitry 120, as discussed above, may be configured to generate a light drive signal that is provided to light source 130 of sensor 102. The light drive signal may, for example, control the intensity of light source 130 and the timing of when light source 130 is turned on and off. In some embodiments, the intensity of light source 130 may be set based on a gain setting in light drive circuitry 120. When light source 130 is configured to emit two or more wavelengths of light, the light drive signal may be configured to control the operation of each wavelength of light. The light drive signal may comprise a single signal or may comprise multiple signals (e.g., one signal for each wavelength of light). An illustrative light drive signal is shown in FIG. 2A.

In some embodiments, control circuitry 110 and light drive circuitry 120 may generate light drive parameters based on a metric. For example, back end processing 170 may receive information about received light signals, determine light drive parameters based on that information, and send corresponding information to control circuitry 110.

FIG. 2A shows an illustrative plot of a light drive signal including red light drive pulse 202 and IR light drive pulse 204 in accordance with some embodiments of the present disclosure. Light drive pulses 202 and 204 are illustrated as square waves. These pulses may include shaped waveforms rather than a square wave. The shape of the pulses may be generated by a digital signal generator, digital filters, analog filters, any other suitable equipment, or any combination thereof. For example, light drive pulses 202 and 204 may be generated by light drive circuitry 120 under the control of control circuitry 110. As used herein, drive pulses may refer to the high and low states of a shaped pulse, switching power or other components on and off, high and low output states, high and low values within a continuous modulation, other suitable relatively distinct states, or any combination thereof. The light drive signal may be provided to light source 130, including red light drive pulse 202 and IR light drive pulse 204 to drive red and IR light emitters, respectively, within light source 130. Red light drive pulse 202 may have a higher amplitude than IR light drive pulse 204 since red LEDs may be less efficient than IR LEDs at converting electrical energy into light energy. In some embodiments, the output levels may be equal, may be adjusted for nonlinearity of emitters, may be modulated in any other suitable technique, or any combination thereof. Additionally, red light may be absorbed and scattered more than IR light when passing through perfused tissue.

When the red and IR light sources are driven in this manner they emit pulses of light at their respective wavelengths into the tissue of a subject in order generate sensor signals that include physiological information that physiological monitoring system 100 may process to calculate physiological parameters. It will be understood that the light drive amplitudes of FIG. 2A are merely exemplary and that any suitable amplitudes or combination of amplitudes may be used, and may be based on the light sources, the subject tissue, the determined physiological parameter, modulation techniques, power sources, any other suitable criteria, or any combination thereof.

The light drive signal of FIG. 2A may also include “off” periods 220 between the red and IR light drive pulses. “Off” periods 220 are periods during which no drive current may be applied to light source 130. “Off” periods 220 may be provided, for example, to prevent overlap of the emitted light, since light source 130 may require time to turn completely on and completely off. The period from time 216 to time 218 may be referred to as a drive cycle, which includes four segments: a red light drive pulse 202, followed by an “off” period 220, followed by an IR light drive pulse 204, and followed by an “off” period 220. After time 218, the drive cycle may be repeated (e.g., as long as a light drive signal is provided to light source 130). It will be understood that the starting point of the drive cycle is merely illustrative and that the drive cycle can start at any location within FIG. 2A, provided the cycle spans two drive pulses and two “off” periods. Thus, each red light drive pulse 202 and each IR light drive pulse 204 may be understood to be surrounded by two “off” periods 220. “Off” periods may also be referred to as dark periods, in that the emitters are dark or returning to dark during that period. It will be understood that the particular square pulses illustrated in FIG. 2A are merely exemplary and that any suitable light drive scheme is possible. For example, light drive schemes may include shaped pulses, sinusoidal modulations, time division multiplexing other than as shown, frequency division multiplexing, phase division multiplexing, any other suitable light drive scheme, or any combination thereof.

Referring back to FIG. 1, front end processing circuitry 150 may receive a detection signal from detector 140 and provide one or more processed signals to back end processing circuitry 170. The term “detection signal,” as used herein, may refer to any of the signals generated within front end processing circuitry 150 as it processes the output signal of detector 140. Front end processing circuitry 150 may perform various analog and digital processing of the detector signal. One suitable detector signal that may be received by front end processing circuitry 150 is shown in FIG. 2B.

FIG. 2B shows an illustrative plot of detector current waveform 214 that may be generated by a sensor in accordance with some embodiments of the present disclosure. The peaks of detector current waveform 214 may represent current signals provided by a detector, such as detector 140 of FIG. 1, when light is being emitted from a light source. The amplitude of detector current waveform 214 may be proportional to the light incident upon the detector. The peaks of detector current waveform 214 may be synchronous with drive pulses driving one or more emitters of a light source, such as light source 130 of FIG. 1. For example, detector current peak 226 may be generated in response to a light source being driven by red light drive pulse 202 of FIG. 2A, and peak 230 may be generated in response to a light source being driven by IR light drive pulse 204. Valleys 228 of detector current waveform 214 may be synchronous with periods of time during which no light is being emitted by the light source, or the light source is returning to dark, such as “off” periods 220. While no light is being emitted by a light source during the valleys, detector current waveform 214 may not fall all of the way to zero.

It will be understood that detector current waveform 214 may be an at least partially idealized representation of a detector signal, assuming perfect light signal generation, transmission, and detection. It will be understood that an actual detector current will include amplitude fluctuations, frequency deviations, droop, overshoot, undershoot, rise time deviations, fall time deviations, other deviations from the ideal, or any combination thereof. It will be understood that the system may shape the drive pulses shown in FIG. 2A in order to make the detector current as similar as possible to idealized detector current waveform 214.

Referring back to FIG. 1, front end processing circuitry 150, which may receive a one or more detection signals, such as detector current waveform 214, may include analog conditioning 152, analog-to-digital converter (ADC) 154, demultiplexer 156, digital conditioning 158, decimator/interpolator 160, and ambient subtractor 162.

Analog conditioning 152 may perform any suitable analog conditioning of the detector signal. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. In some embodiments, one or more gain settings may be used in analog conditioning 152 to adjust the amplification of detector signal.

The conditioned analog signal may be processed by analog-to-digital converter 154, which may convert the conditioned analog signal into a digital signal. Analog-to-digital converter 154 may operate under the control of control circuitry 110. Analog-to-digital converter 154 may use timing control signals from control circuitry 110 to determine when to sample the analog signal. Analog-to-digital converter 154 may be any suitable type of analog-to-digital converter of sufficient resolution to enable a physiological monitor to accurately determine physiological parameters.

Demultiplexer 156 may operate on the analog or digital form of the detector signal to separate out different components of the signal. For example, detector current waveform 214 of FIG. 2B includes a red component corresponding to peak 226, an IR component corresponding to peak 230, and at least one ambient component corresponding to valleys 228. Demultiplexer 156 may operate on detector current waveform 214 of FIG. 2B to generate a red signal, an IR signal, a first ambient signal (e.g., corresponding to the ambient component corresponding to valley 228 that occurs immediately after the peak 226), and a second ambient signal (e.g., corresponding to the ambient component corresponding to valley 232 that occurs immediately after peak 230). Demultiplexer 156 may operate under the control of control circuitry 110. For example, demultiplexer 156 may use timing control signals from control circuitry 110 to identify and separate out the different components of the detector signal.

Digital conditioning 158 may perform any suitable digital conditioning of the detector signal. Digital conditioning 158 may include any type of digital filtering of the signal (e.g., low pass, high pass, band pass, notch, or any other suitable filtering), amplifying, performing an operation on the signal, performing any other suitable digital conditioning, or any combination thereof.

Decimator/interpolator 160 may decrease the number of samples in the digital detector signal. For example, decimator/interpolator 160 may decrease the number of samples by removing samples from the detector signal or replacing samples with a smaller number of samples. The decimation or interpolation operation may include or be followed by filtering to smooth the output signal.

Ambient subtractor 162 may operate on the digital signal. In some embodiments, ambient subtractor 162 may remove dark or ambient contributions to the received signal or signals.

The components of front end processing circuitry 150 are merely illustrative and any suitable components and combinations of components may be used to perform the front end processing operations.

The front end processing circuitry 150 may be configured to take advantage of the full dynamic range of analog-to-digital converter 154. This may be achieved by applying one or more gains to the detection signal, by analog conditioning 152 to map the expected range of the signal to the full or close to full output range of analog-to-digital converter 154. The output value of analog-to-digital converter 154, as a function of the total analog gain applied to the detection signal, may be given as:

ADC Value=Total Analog Gain×[Ambient Light+LED Light]

Ideally, when ambient light is zero and when the light source is off, the analog-to-digital converter 154 will read just above the minimum input value. When the light source is on, the total analog gain may be set such that the output of analog-to-digital converter 154 may read close to the full scale of analog-to-digital converter 154 without saturating. This may allow the full dynamic range of analog-to-digital converter 154 to be used for representing the detection signal, thereby increasing the resolution of the converted signal. In some embodiments, the total analog gain may be reduced by a small amount so that small changes in the light level incident on the detector do not cause saturation of analog-to-digital converter 154.

However, if the contribution of ambient light is large relative to the contribution of light from a light source, the total analog gain applied to the detection current may need to be reduced to avoid saturating analog-to-digital converter 154. When the analog gain is reduced, the portion of the signal corresponding to the light source may map to a smaller number of analog-to-digital conversion bits. Thus, more ambient light noise in the input of analog-to-digital converter 154 may results in fewer bits of resolution for the portion of the signal from the light source. This may have a detrimental effect on the signal-to-noise ratio of the detection signal. Accordingly, passive or active filtering or signal modification techniques may be employed to reduce the effect of ambient light on the detection signal that is applied to analog-to-digital converter 154, and thereby reduce the contribution of the noise component to the converted digital signal.

Back end processing circuitry 170 may include processor 172 and memory 174. Processor 172 may be adapted to execute software, which may include an operating system and one or more applications, as part of performing the functions described herein. Processor 172 may receive and further process sensor signals received from front end processing circuitry 150. For example, processor 172 may determine one or more physiological parameters based on the received physiological signals. Processor 172 may include an assembly of analog or digital electronic components. Processor 172 may calculate physiological information. For example, processor 172 may compute one or more of fluid responsiveness, a blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processor 172 may perform any suitable signal processing of a signal, such as any suitable scaling, band-pass filtering, adaptive filtering, closed-loop filtering, any other suitable filtering, and/or any combination thereof. Processor 172 may also receive input signals from additional sources not shown. For example, processor 172 may receive an input signal containing information about treatments provided to the subject from user interface 180. Additional input signals may be used by processor 172 in any of the calculations or operations it performs in accordance with back end processing circuitry 170 or monitor 104.

Memory 174 may include any suitable computer-readable media capable of storing information that can be interpreted by processor 172. In some embodiments, memory 174 may store calculated values, such as pulse rate, blood pressure, blood oxygen saturation, fiducial point locations or characteristics, initialization parameters, systemic vascular resistance, mean arterial pressure, cardiac output, central venous pressure, oxygen demand, adaptive filter parameters, fluid responsiveness parameters, any other calculated values, or any combination thereof, in a memory device for later retrieval. This information may be data or may take the form of computer-executable instructions, such as software applications, that cause a microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system. Back end processing circuitry 170 may be communicatively coupled with user interface 180 and communication interface 190.

User interface 180 may include user input 182, display 184, and speaker 186. User interface 180 may include, for example, any suitable device such as one or more medical devices (e.g., a medical monitor that displays various physiological parameters, a medical alarm, or any other suitable medical device that either displays physiological parameters or uses the output of back end processing 170 as an input), one or more display devices (e.g., monitor, personal digital assistant (PDA), mobile phone, tablet computer, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

User input 182 may include any type of user input device such as a keyboard, a mouse, a touch screen, buttons, switches, a microphone, a joy stick, a touch pad, or any other suitable input device. The inputs received by user input 182 can include information about the subject, such as age, weight, height, diagnosis, medications, treatments, and so forth.

In an embodiment, the subject may be a medical patient and display 184 may exhibit a list of values which may generally apply to the patient, such as, for example, age ranges or medication families, which the user may select using user input 182. Additionally, display 184 may display, for example, an estimate of a subject's blood oxygen saturation generated by monitor 104 (e.g., an “SpO2” or a regional oximetry measurement), fluid responsiveness information, pulse rate information, respiration rate and/or effort information, blood pressure information, hemoglobin concentration information, systemic vascular resistance, mean arterial pressure, cardiac output, central venous pressure, oxygen demand, any other parameters, and any combination thereof. Display 184 may include any type of display such as a cathode ray tube display, a flat panel display such as a liquid crystal display or plasma display, or any other suitable display device. Speaker 186 within user interface 180 may provide an audible sound that may be used in various embodiments, such as for example, sounding an audible alarm in the event that a patient's physiological parameters are not within a predefined normal range.

Communication interface 190 may enable monitor 104 to exchange information with external devices. Communications interface 190 may include any suitable hardware, software, or both, which may allow monitor 104 to communicate with electronic circuitry, a device, a network, a server or other workstations, a display, or any combination thereof. Communications interface 190 may include one or more receivers, transmitters, transceivers, antennas, plug-in connectors, ports, communications buses, communications protocols, device identification protocols, any other suitable hardware or software, or any combination thereof. Communications interface 190 may be configured to allow wired communication (e.g., using USB, RS-232, Ethernet, or other standards), wireless communication (e.g., using WiFi, IR, WiMax, BLUETOOTH, USB, or other standards), or both. For example, communications interface 190 may be configured using a universal serial bus (USB) protocol (e.g., USB 2.0, USB 3.0), and may be configured to couple to other devices (e.g., remote memory devices storing templates) using a four-pin USB standard Type-A connector (e.g., plug and/or socket) and cable. In some embodiments, communications interface 190 may include an internal bus such as, for example, one or more slots for insertion of expansion cards.

It will be understood that the components of physiological monitoring system 100 that are shown and described as separate components are shown and described as such for illustrative purposes only. In some embodiments the functionality of some of the components may be combined in a single component. For example, the functionality of front end processing circuitry 150 and back end processing circuitry 170 may be combined in a single processor system. Additionally, in some embodiments the functionality of some of the components of monitor 104 shown and described herein may be divided over multiple components. For example, some or all of the functionality of control circuitry 110 may be performed in front end processing circuitry 150, in back end processing circuitry 170, or both. In other embodiments, the functionality of one or more of the components may be performed in a different order or may not be required. In an embodiment, all of the components of physiological monitoring system 100 can be realized in processor circuitry.

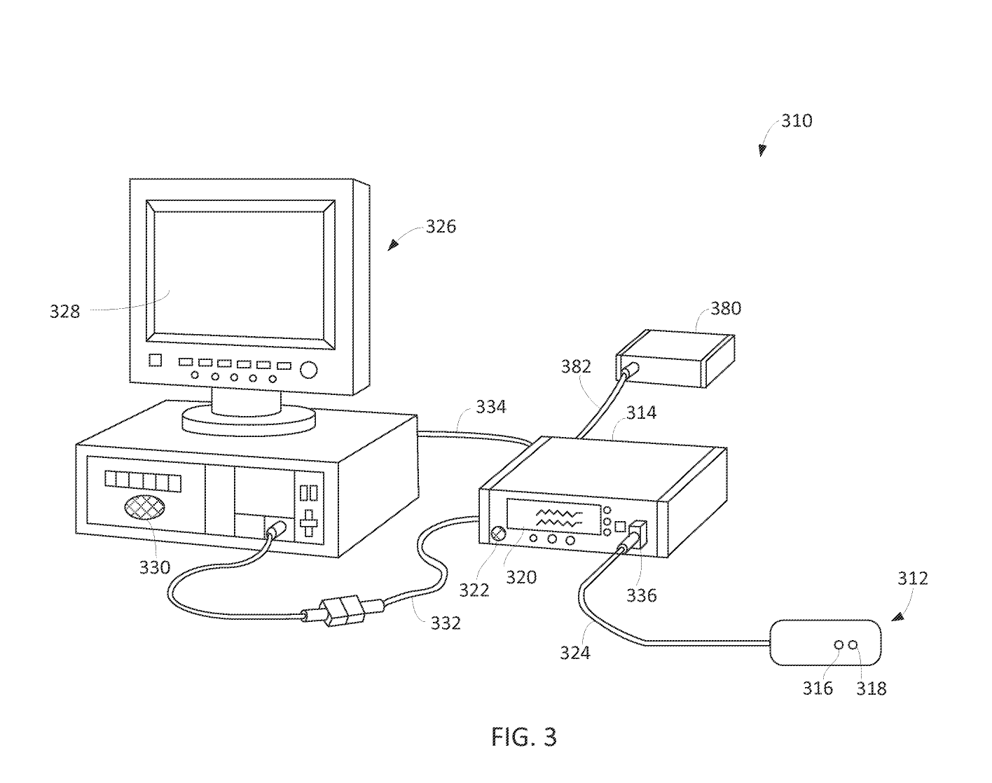

FIG. 3 is a perspective view of an illustrative physiological monitoring system 310 in accordance with some embodiments of the present disclosure. In some embodiments, one or more components of physiological monitoring system 310 may include one or more components of physiological monitoring system 100 of FIG. 1. Physiological monitoring system 310 may include sensor unit 312 and monitor 314. In some embodiments, sensor unit 312 may be part of an oximeter. Sensor unit 312 may include one or more light source 316 for emitting light at one or more wavelengths into a subject's tissue. One or more detector 318 may also be provided in sensor unit 312 for detecting the light that is reflected by or has traveled through the subject's tissue. Any suitable configuration of light source 316 and detector 318 may be used. In an embodiment, sensor unit 312 may include multiple light sources and detectors, which may be spaced apart. Physiological monitoring system 310 may also include one or more additional sensor units (not shown) that may, for example, take the form of any of the embodiments described herein with reference to sensor unit 312. An additional sensor unit may be the same type of sensor unit as sensor unit 312, or a different sensor unit type than sensor unit 312 (e.g., a photoacoustic sensor). Multiple sensor units may be capable of being positioned at two different locations on a subject's body. In an example, an oximeter sensor may be located at a first position and a thermodilution sensor may be located at a second location. In another example, an oximeter sensor and a temperature sensor may be located near to one another or in the same structure.

In some embodiments, sensor unit 312 may be connected to monitor 314 as shown. Sensor unit 312 may be powered by an internal power source, e.g., a battery (not shown). Sensor unit 312 may draw power from monitor 314. In another embodiment, the sensor may be wirelessly connected (not shown) to monitor 314. Monitor 314 may be configured to calculate physiological parameters based at least in part on data relating to light emission and light detection received from one or more sensor units such as sensor unit 312. For example, monitor 314 may be configured to determine fluid responsiveness, pulse rate, respiration rate, respiration effort, blood pressure, blood oxygen saturation (e.g., arterial, venous, regional, or a combination thereof), hemoglobin concentration (e.g., oxygenated, deoxygenated, and/or total), systemic vascular resistance, mean arterial pressure, cardiac output, central venous pressure, oxygen demand, any other suitable physiological parameters, or any combination thereof. In some embodiments, calculations may be performed on the sensor units or an intermediate device and the result of the calculations may be passed to monitor 314. Further, monitor 314 may include display 320 configured to display the physiological parameters or other information about the system. In the embodiment shown, monitor 314 may also include a speaker 322 to provide an audible sound that may be used in various other embodiments, such as for example, sounding an audible alarm in the event that a subject's physiological parameters are not within a predefined normal range. In some embodiments, physiological monitoring system 310 may include a stand-alone monitor in communication with the monitor 314 via a cable or a wireless network link. In some embodiments, monitor 314 may be implemented as display 184 of FIG. 1.

In some embodiments, sensor unit 312 may be communicatively coupled to monitor 314 via a cable 324 at port 336. Cable 324 may include electronic conductors (e.g., wires for transmitting electronic signals from detector 318), optical fibers (e.g., multi-mode or single-mode fibers for transmitting emitted light from light source 316), any other suitable components, any suitable insulation or sheathing, or any combination thereof. In some embodiments, a wireless transmission device (not shown) or the like may be used instead of or in addition to cable 324. Monitor 314 may include a sensor interface configured to receive physiological signals from sensor unit 312, provide signals and power to sensor unit 312, or otherwise communicate with sensor unit 312. The sensor interface may include any suitable hardware, software, or both, which may be allow communication between monitor 314 and sensor unit 312.

In some embodiments, physiological monitoring system 310 may include calibration device 380. Calibration device 380, which may be powered by monitor 314, a battery, or by a conventional power source such as a wall outlet, may include any suitable calibration device. Calibration device 380 may be communicatively coupled to monitor 314 via communicative coupling 382, and/or may communicate wirelessly (not shown). In some embodiments, calibration device 380 is completely integrated within monitor 314. In some embodiments, calibration device 380 may include a manual input device (not shown) used by an operator to manually input reference signal measurements obtained from some other source (e.g., an external invasive or non-invasive physiological measurement system).

In the illustrated embodiment, physiological monitoring system 310 includes a multi-parameter physiological monitor 326. The monitor 326 may include a cathode ray tube display, a flat panel display (as shown) such as a liquid crystal display (LCD) or a plasma display, or may include any other type of monitor now known or later developed. Multi-parameter physiological monitor 326 may be configured to calculate physiological parameters and to provide a display 328 for information from monitor 314 and from other medical monitoring devices or systems (not shown). For example, multi-parameter physiological monitor 326 may be configured to display an estimate of a subject's fluid responsiveness, blood oxygen saturation, and hemoglobin concentration generated by monitor 314. Multi-parameter physiological monitor 326 may include a speaker 330.

Monitor 314 may be communicatively coupled to multi-parameter physiological monitor 326 via a cable 332 or 334 that is coupled to a sensor input port or a digital communications port, respectively and/or may communicate wirelessly (not shown). In addition, monitor 314 and/or multi-parameter physiological monitor 326 may be coupled to a network to enable the sharing of information with servers or other workstations (not shown). Monitor 314 may be powered by a battery (not shown) or by a conventional power source such as a wall outlet.

In some embodiments, any of the processing components and/or circuits, or portions thereof, of FIGS. 1 and 3, including sensors 102 and 312 and monitors 104, 314, and 326, may be referred to collectively as processing equipment. For example, processing equipment may be configured to amplify, filter, sample and digitize an input signal from sensor 102 or 312 (e.g., using an analog-to-digital converter), and calculate physiological information from the digitized signal. Processing equipment may be configured to generate light drive signals, amplify, filter, sample and digitize detector signals, sample and digitize other analog signals, calculate physiological information from the digitized signal, perform any other suitable processing, or any combination thereof. In some embodiments, all or some of the components of the processing equipment may be referred to as a processing module.

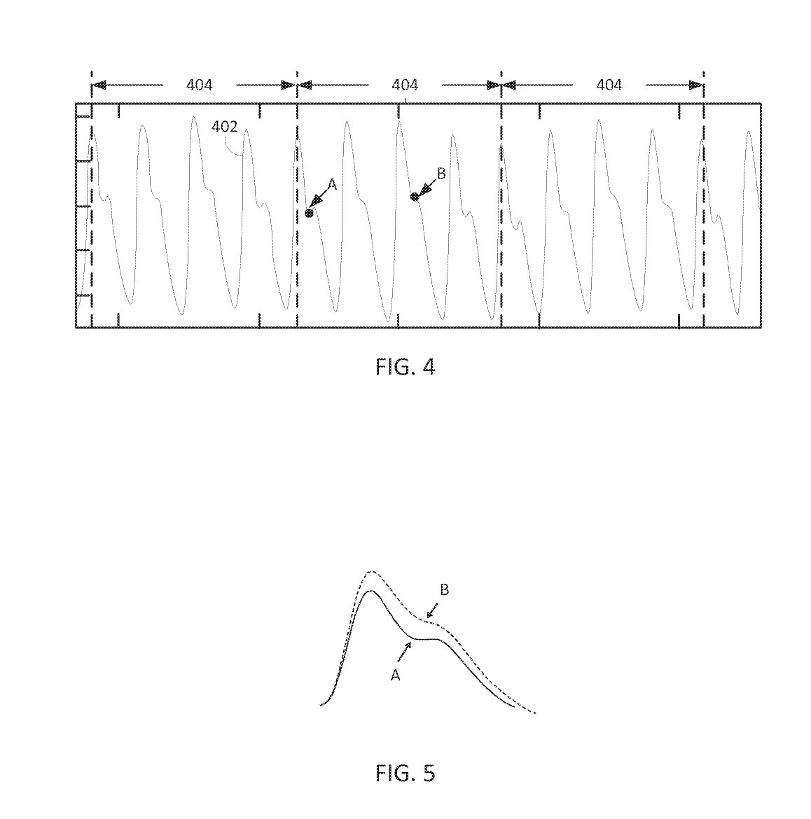

FIG. 4 shows an illustrative PPG signal 402 that is modulated by respiration in accordance with some embodiments of the present disclosure. PPG signal 402 may be a periodic signal that is indicative of changes in pulsatile blood flow. Each cycle of PPG signal 402 may generally correspond to a pulse, such that a heart rate may be determined based on PPG signal 402. Each respiratory cycle 404 may correspond to a breath. The period of a respiratory cycle may typically be longer than the period of a pulsatile cycle, such that any changes in the pulsatile blood flow due to respiration occur over a number of pulsatile cycles. The volume of the pulsatile blood flow may also vary in a periodic manner based on respiration, resulting in modulations to the pulsatile blood flow such as amplitude modulation, frequency modulation, and baseline modulation. This modulation of PPG signal 402 due to respiration may result in changes to the morphology of PPG signal 402.

FIG. 5 shows a comparison of portions of the illustrative PPG signal 402 of FIG. 4 in accordance with some embodiments of the present disclosure. The signal portions compared in FIG. 5 may demonstrate differing morphology due to respiration modulation based on the relative location of the signal portions within a respiratory cycle 404. For example, a first pulse associated with the respiratory cycle may have a relatively low amplitude (indicative of amplitude and baseline modulation) as well as an obvious distinct dichrotic notch as indicated by point A. A second pulse may have a relatively high amplitude (indicative of amplitude and baseline modulation) as well as a dichrotic notch that has been washed out as depicted by point B. Frequency modulation may be evident based on the relative period of the first pulse and second pulse. Referring again to FIG. 4, by the end of the respiratory cycle 404 the pulse features may again be similar to the morphology of A. Although the impact of respiration modulation on the morphology of a particular PPG signal 402 has been described herein, it will be understood that respiration may have varied effects on the morphology of a PPG signal other than those depicted in FIGS. 4 and 5.

In accordance with some embodiments of the present disclosure, respiration morphology signals may be generated from a PPG signal. In some embodiments, a plurality of respiration morphology signals may be generated from the PPG signal, and the plurality of respiration morphology signals may be selected. Although respiration morphology signals may be generated in any suitable manner, in an exemplary embodiment, respiration morphology signals may be generated based on calculating a series of morphology metrics based on a PPG signal. One or more morphology metrics maybe calculated for each portion of the PPG signal (e.g., for each fiducial defined portion), a series of morphology metrics may be calculated over time, and the series of morphology metrics may be processed to generate one or more respiration morphology signals.

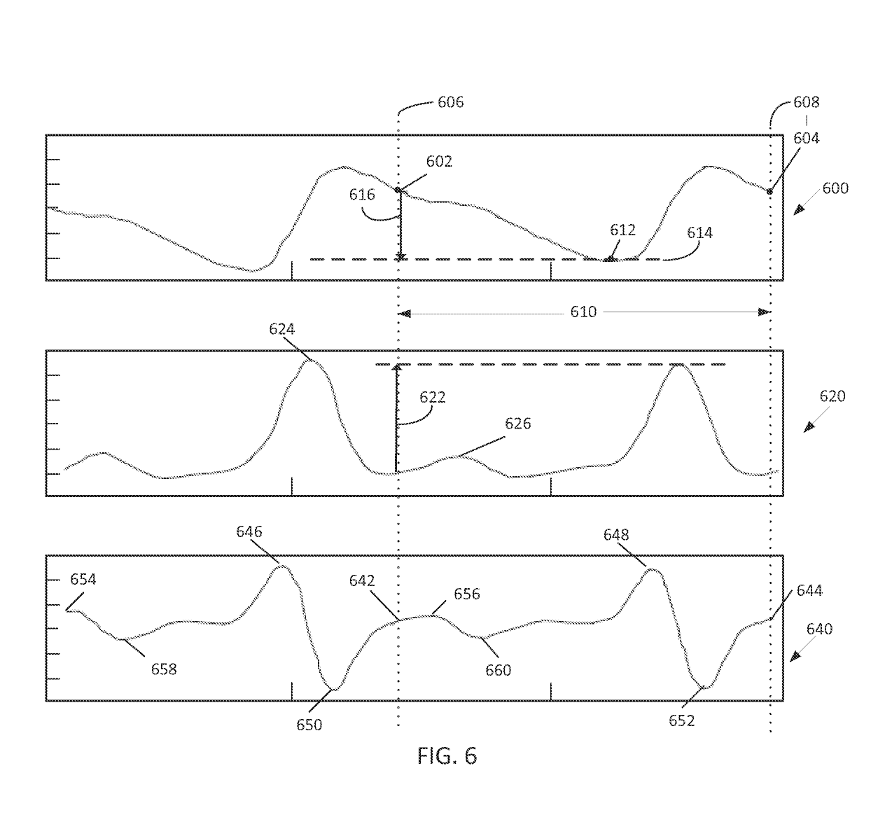

FIG. 6 depicts exemplary signals used for calculating morphology metrics from a received PPG signal. The abscissa of each plot of FIG. 6 may represent time and the ordinate of each plot may represent magnitude. PPG signal 600 may be a received PPG signal, first derivative signal 620 may be a signal representing the first derivative of the PPG signal 600, and second derivative signal 640 may be a signal representing the second derivative of the PPG signal 600. As will be described herein, morphology metrics may be calculated for portions of these signals, and a series of morphology metric calculations calculated over time may be processed to generate the respiration morphology signals. Although particular morphology metric calculations are set forth below, each of the morphology metric calculations may be modified in any suitable manner.

Although morphology metrics may be calculated based on any suitable portions of the PPG signal 600 (as well as the first derivative signal 620, second derivative signal 640, and any other suitable signals that may be generated from the PPG signal 600), in an exemplary embodiment, morphology metrics may be calculated for each fiducial-defined portion such as fiducial defined portion 610 of the PPG signal 600. Exemplary fiducial points 602 and 604 are depicted for PPG signal 600, and fiducial lines 606 and 608 demonstrate the location of fiducial points 602 and 604 relative to first derivative signal 620 and second derivative signal 640.

Although it will be understood that fiducial points may be identified in any suitable manner, in exemplary embodiments fiducial points may be identified based on features of the PPG signal 620 or any derivatives thereof (e.g., first derivative signal 620 and second derivative signal 640) such as peaks, troughs, points of maximum slope, dichrotic notch locations, pre-determined offsets, any other suitable features, or any combination thereof. Fiducial points 602 and 604 may define a fiducial-defined portion 610 of PPG signal 600. The fiducial points 602 and 604 may define starting and ending points for determining morphology metrics, and the fiducial-defined portion 610 may define a relevant portion of data for determining morphology metrics. It will be understood that other starting points, ending points, and relative portions of data may be utilized to determine morphology metrics.

An exemplary morphology metric may be a down metric. The down metric is the difference between a first (e.g., fiducial) sample of a fiducial-defined portion (e.g., fiducial defined portion 610) of the PPG signal (e.g., PPG signal 600) and a minimum sample (e.g., minimum sample 612) of the fiducial-defined portion 610 of the PPG signal 600. The down metric may also be calculated based on other points of a fiducial-defined portion. The down metric is indicative of physiological characteristics which are related to respiration, e.g., amplitude and baseline modulations of the PPG signal. In an exemplary embodiment, fiducial point 602 defines the first location for calculation of a down metric for fiducial-defined portion 610. In the exemplary embodiment, the minimum sample of fiducial-defined portion 610 is minimum point 612, and is indicated by horizontal line 614. The down metric may be calculated by subtracting the value of minimum point 612 from the value of fiducial point 602, and is depicted as down metric 616.

Another exemplary morphology metric may be a kurtosis metric for a fiducial-defined portion. Kurtosis measures the peakedness of the PPG signal 600 or a derivative thereof (e.g., first derivative signal 620 or second derivative signal 640). In an exemplary embodiment, the kurtosis metric may be based on the peakedness of the first derivative signal 620. The peakedness is sensitive to both amplitude and period (frequency) changes, and may be utilized as an input to generate respiration morphology signals that may be used to determine respiration information such as respiration rate. Kurtosis may be calculated based on the following formulae:

$$D = \frac{1}{n} {\sum }_{i = 1}^{n} {( {x}_{i}^{′} - {\overline{x}}^{′} )}^{2}$$

$$Kurtosis = \frac{1}{{nD}^{2}} {\sum }_{i = 1}^{n} {( {x}_{i}^{′} - {\overline{x}}^{′} )}^{4}$$

where:
xᵢ′=ith sample of 1^{st} derivative; x̅′̅=mean of 1st derivative of fiducial-defined portion;
n=set of all samples in the fiducial-defined portion

Another exemplary morphology metric may be a delta of the second derivative (DSD) between consecutive fiducial-defined portions, e.g., at consecutive fiducial points. Measurement points 642 and 644 for a DSD calculation are depicted at fiducial points 602 and 604 as indicated by fiducial lines 606 and 608. The second derivative signal is indicative of the curvature of a signal. Changes in the curvature of the PPG signal 600 that can be identified with second derivative signal 640 are indicative of changes in internal pressure that occur during respiration, particularly changes near the peak of a pulse. By providing a metric of changes in curvature of the PPG signal, the DSD morphology metric may be utilized as an input to determine respiration information, such as respiration rate. The DSD metric may be calculated for each fiducial-defined portion by identifying the value of the second derivative signal 640 at the current fiducial point (e.g., fiducial point 642 of fiducial-defined portion 610) and subtracting from that the value of the second derivative signal 640 at the next fiducial point (e.g., fiducial point 644 of fiducial-defined portion 610).

Another exemplary morphology metric may be an up metric measuring the up stroke of the first derivative signal 620 of the light intensity signal. The up stroke may be based on an initial starting sample (fiducial point) and a maximum sample for the fiducial-defined portion and is depicted as up metric 622 for a fiducial point corresponding to fiducial line 606. The up metric may be indicative of amplitude and baseline modulation of the light intensity signal, which may be related to respiration information as described herein. Although an up metric is described herein with respect to the first derivate signal 620, it will be understood that an up metric may also be calculated for the light intensity signal 600 and second derivative signal 640.

Another exemplary morphology metric may be a skew metric measuring the skewness of the original light intensity signal 600 or first derivative 620. The skewness metric is indicative of amplitude and frequency modulation of the light intensity signal, which may be related to respiration information as described herein. Skewness may be calculated as follows:

$$\begin{matrix}g 1 = \frac{{m}_{3}}{{m}_{2}^{3 / 2}} \\ = \frac{\frac{1}{n} {\sum }_{i = 1}^{n} {( {x}_{i}^{′} - {\overline{x}}^{′} )}^{3}}{{( \frac{1}{n} {\sum }_{i = 1}^{n} {( {x}_{i} - {\overline{x}}^{′} )}^{2} )}^{3 / 2}}\end{matrix}$$

where:
xᵢ=ith sample;
x̅=mean of the samples of the fiducial-defined portion;
m₃=third moment;
m₂=second moment; and
n=total number of samples.

Another exemplary morphology metric may be a b/a ratio metric (i.e., b/a), which is based on the ratio between the a-peak and b-peak of the second derivative signal 640. Light intensity signal 600, first derivative signal 620, and second derivative signal 640 may include a number of peaks (e.g., four peaks corresponding to maxima and minima) which may be described as the a-peak, b-peak, c-peak, and d-peak, with the a-peak and c-peak generally corresponding to local maxima within a fiducial defined portion and the b-peak and d-peak generally corresponding to local minima within a fiducial defined portion. For example, the second derivative of the light intensity signal may include four peaks: the a-peak, b-peak, c-peak, and d-peak. Each peak may be indicative of a respective systolic wave, i.e., the a-wave, b-wave, c-wave, and d-wave. On the depicted portion of the second derivative of the light intensity signal 640, the a-peaks are indicated by points 646 and 648, the b-peaks by points 650 and 652, the c-peaks by points 654 and 656, and the d-peaks by points 658 and 660. The b/a ratio measures the ratio of the b-peak (e.g., 650 or 652) and the a-peak (e.g., 646 or 648). The b/a ratio metric may be indicative of the curvature of the light intensity signal, which demonstrates frequency modulation based on respiration information such as respiration rate. The b/a ratio may also be calculated based on the a-peak and b-peak in higher order signals such as light intensity signal and first derivative light intensity signal 620.

Another exemplary morphology metric may be a c/a ratio (i.e., c/a), which is calculated from the a-peak and c-peak of a signal. For example, first derivate light intensity signal 620 may have a c-peak 626 which corresponds to the maximum slope near the dichrotic notch of light intensity signal 600, and an a-peak 624 which corresponds to the maximum slope of the light intensity signal 600. The c/a ratio of the first derivative is indicative of frequency modulation of the light intensity signal, which is related to respiration information such as respiration rate as described herein. A c/a ratio may be calculated in a similar manner for light intensity signal 600 and second derivative signal 640.

Another exemplary morphology metric may be a i_b metric measuring the time between two consecutive local minimum (b) locations 650 and 652 in the second derivative 640. The i_b metric is indicative of frequency modulation of the light intensity signal, which is related to respiration information such as respiration rate as described herein. The i_b metric may also be calculated for light intensity signal 600 or first derivative signal 620.

Another exemplary morphology metric may be a peak amplitude metric measuring the amplitude of the peak of the original light intensity signal 600 or of the higher order derivatives 620 and 640. The peak amplitude metric is indicative of amplitude modulation of the light intensity signal, which is related to respiration information such as respiration rate as described herein.

Another exemplary morphology metric may be a center of gravity metric measuring the center of gravity of a fiducial-defined portion from the light intensity signal 600 in either or both of the x and y coordinates. The center of gravity is calculated as follows:

Center of gravity(x)=Σ(xᵢ*yᵢ)/Σyᵢ

Center of gravity(y)=Σ(xᵢ*yᵢ)/Σxᵢ

The center of gravity metric of the x coordinate for a fiducial-defined portion is indicative of frequency modulation of the light intensity signal, which is related to respiration information such as respiration rate as described herein. The center of gravity metric of the y coordinate for a fiducial-defined portion is indicative of amplitude modulation of the light intensity signal, which is related to respiration information such as respiration rate as described herein.

Another exemplary morphology metric is an area metric measuring the total area under the curve for a fiducial-defined portion of the light intensity signal 600. The area metric is indicative of frequency and amplitude modulation of the light intensity signal, which is related to respiration information such as respiration rate as described herein.

Another morphology metric is the light intensity amplitude metric. This metric represents the amplitude of the patient's light intensity signal. In some embodiments, the light intensity amplitude metric is normalized to the baseline (i.e., DC component) of the underlying light intensity signal.

Another morphology metric is the light intensity amplitude modulation metric. This metric represents the modulation of amplitude over time on a patient's light intensity signal.

Another morphology metric is the frequency modulation metric. This metric represents the modulation of periods between fiducial points on a physiological signal, such as a light intensity signal.

Although a number of morphology metrics have been described herein, it will be understood that other morphology metrics may be calculated from light intensity signal 600, first derivative signal 620, second derivative signal 640, and any other order of the light intensity signal. It will also be understood that any of the morphology metrics described above may be modified to capture aspects of respiration information or other physiological information that may be determined from a light intensity signal.

In some embodiments, each series of morphology metric values may be further processed in any suitable manner to generate the respiration morphology signals. Although any suitable processing operations may be performed for each series of morphology metric values, in an exemplary embodiment, each series of morphology metric values may be filtered (e.g., based on frequencies associated with respiration) and interpolated to generate the plurality of respiration morphology signals.

In an embodiment, an autocorrelation sequence may be generated for each of the respiration morphology signals. The peaks of an autocorrelation correspond to portions of the signal that include the same or similar information. Thus, the peaks of the autocorrelation sequences may correspond to periodic aspects of the underlying morphology signals, which in turn may correspond to respiration information such as respiration rate.

In some embodiments, the aforementioned respiration morphology signals may be processed in the manner above, in accordance with the embodiments disclosed in U.S. Patent Application Ser. No. 61/896,581, filed on Oct. 28, 2013, the contents of which are entirely incorporated by reference herein, in any other suitable manner, or any combination thereof to determine respiration rate in a subject.

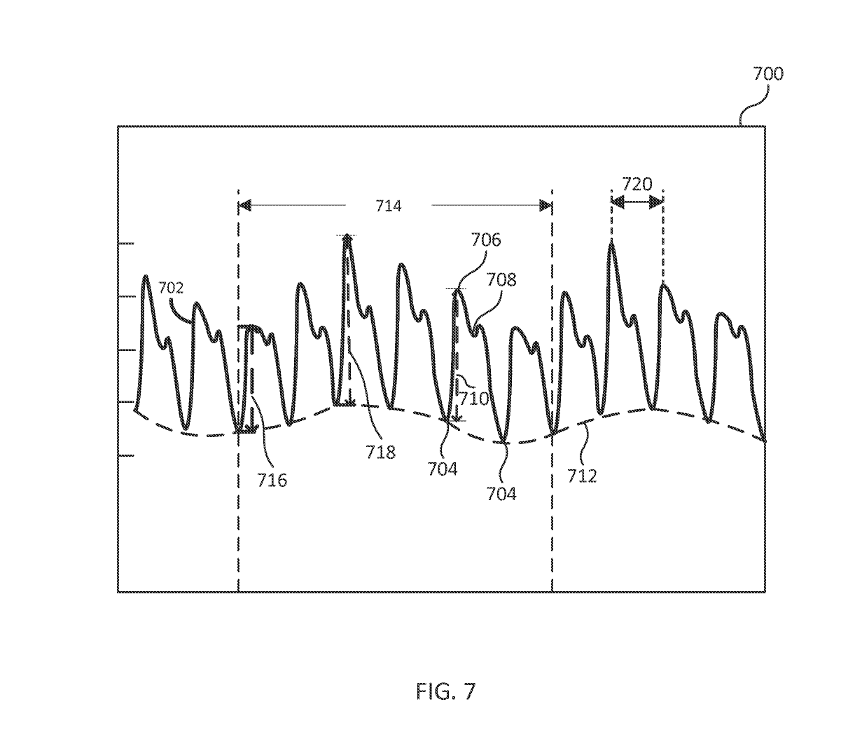

FIG. 7 shows an illustrative plot 700 of PPG waveform 702 reflecting respiratory modulations used to determine fluid responsiveness in accordance with some embodiments of the present disclosure. PPG waveform 702 may be generated, for example, by system 100 of FIG. 1 or system 310 of FIG. 3. As illustrated, PPG waveform 702 represents the absorption of light by a subject's tissue over time. PPG waveform 702 includes pulses where the absorption of light increases due to the increased volume of blood in the arterial blood vessel due to cardiac pulses. In some embodiments, pulses may be identified between adjacent valleys 704 and as illustrated may include a peak 706 and a dichrotic notch 708. The pulses include an upstroke between the first valley and the main peak. For example, an upstroke is depicted in FIG. 7 between the first valley 704 and peak 706. The amplitude of this upstroke is depicted as amplitude 710 measured from the first valley 704 to peak 706. Other amplitude values may be derived from the PPG waveform, such as a downstroke amplitude, average amplitude, or area under the pulse. In some embodiments, the amplitude of a pulse may be determined by subtracting a minimum value of PPG waveform 702 from a maximum value of PPG waveform 702 within a segment of PPG waveform 702 that generally corresponds to the period of a pulse. PPG waveform 702 also includes a varying baseline 712. PPG waveform 702 modulates above baseline 712 due to the pulses.

For most subjects, the PPG signal is affected by the subject's respiration, i.e. inhaling and exhaling, resulting in certain respiration modulations in the PPG waveform. FIG. 7 illustrates respiration modulations in PPG waveform 702 as a result of the subject's inhaling and exhaling. One type of respiratory modulation is the modulation of baseline 712 of PPG waveform 702. The effect of the subject's breathing in and out causes the baseline of the waveform 702 to move up and down, cyclically, with the subject's respiration. The baseline may be tracked by following any fiducial of PPG waveform 702, such as the peaks 706, valleys 704, dichrotic notches 708, median value, or any other fiducials. A second type of respiration-induced modulation of PPG waveform 702 is the modulation of pulse amplitudes. As the patient breathes in and out, the amplitudes of pulses decreases and increases, with larger amplitudes tending to coincide with the top of the baseline shift, and smaller amplitudes tending to coincide with the bottom of the baseline shift (though the larger and smaller amplitudes do not necessarily fall at the top and bottom of the baseline shift). A third respiratory type of modulation is the modulation of period 720 between pulses (also referred to as frequency modulation). Each of these modulations may be referred to as a respiratory component of PPG waveform 702, or a respiratory-induced modulation of PPG waveform 702. It should be noted that a particular individual may exhibit only the baseline modulation, or only the amplitude modulation, or only the frequency modulation, or any combination thereof. As referred to herein, a respiratory component of the PPG waveform 702 includes any one of these respiratory-induced modulations of PPG waveform 702, a measure of these modulations, or a combination of them.

The respiratory modulations of PPG waveform 702 can be affected by a subject's fluid status. For example, a hypovolemic subject may exhibit relatively larger respiratory variations of PPG waveform 702. When a subject loses fluid, the subject may have decreased cardiac output or stroke volume, which tends to increase the respiratory variations present in the subject's PPG waveform. Specifically, the baseline modulation, amplitude modulation, and frequency modulation may become more pronounced. Thus, larger respiratory modulations may indicate that the subject will respond favorably to fluid loading, whereas smaller respiratory modulations may indicate that a patient may not respond favorably to fluid loading. The respiratory modulations of the PPG waveform 702, may be identified and used to determine a subject's fluid responsiveness.

In some embodiments, a physiological monitor receives a PPG signal and determines fluid responsiveness based on the PPG signal. In some embodiments, the fluid responsiveness is a measure of a subject's likely response to fluid therapy. In some embodiments, the fluid responsiveness is a metric that reflects a degree of respiratory variation of the PPG signal. One such example of fluid responsiveness is a measure of the amplitude modulations of the PPG signal, such as Delta POP (DPOP or ΔPOP, defined below). Another example of a parameter indicative of fluid responsiveness is a measure of the baseline modulation of the PPG. In some embodiments, other suitable metrics or combinations of metrics may be used to assess the respiratory modulation of the PPG signal. For example, a parameter indicative of fluid responsiveness may be based on the amplitudes or areas of acceptable pulses within a particular time frame or window. For example, as illustrated in FIG. 7, minimum amplitude 716 of the pulses within respiratory period 714 may be subtracted from maximum amplitude 718 within respiratory period 714 and then divided by an average or mean value of minimum amplitude 716 and maximum amplitude 718. In some embodiments, fluid responsiveness may be derived from the period or frequency of pulses within a time frame or window. For example, a modulation or variation in the period or frequency among two or more cardiac pulses may be used to derive fluid responsiveness. In general, fluid responsiveness may be based on one or more respiratory variations exhibited by the PPG waveform 702. Further, fluid responsiveness may be determined through the use of wavelet transforms, such as described in United States Patent Application Publication No. 2010/0324827, entitled “Fluid Responsiveness Measure,” which is hereby incorporated by reference in its entirety.

In some embodiments, DPOP is used as the measure of fluid responsiveness. The DPOP metric can be calculated from PPG waveform 702 for a particular time window as follows:

DPOP=(AMPₘₐₓ−AMPₘᵢₙ)/AMPₐᵥₑ (1)

where AMPₘₐₓ represents the maximum amplitude (such as maximum amplitude 718 in FIG. 7) during a time window (such as respiratory period 714 in FIG. 7), AMPₘᵢₙ represents the minimum amplitude (such as minimum amplitude 716 in FIG. 7) during the time window, and AMPₐᵥₑ is the average of the two, as follows:

AMPₐᵥₑ=(AMPₘₐₓ+AMPₘᵢₙ)/2 (2)

In some embodiments, AMPₘₐₓ and AMPₘᵢₙ may be measured at other locations of the PPG, such as within or along a pulse. DPOP is a measure of the respiratory variation in the AC portion of the PPG signal. DPOP is a unit-less value, and in some embodiments can be expressed as a percentage. In some embodiments, respiratory period 714 is one respiratory cycle (inhalation and exhalation). In some embodiments, respiratory period 714 is a fixed duration of time that approximates one respiratory cycle, such as 5 seconds, 10 seconds, or any other suitable duration. In some embodiments, respiratory period 714 may be adjusted dynamically based on the subject's calculated or measured respiration rate, so that the period is approximately the same as one respiratory cycle period. In some embodiments, a signal turning point detector may be used to identify the maximum and minimum points in the PPG signal, in order to calculate the upstroke amplitudes.

In some embodiments, it is desirable to determine fluid responsiveness by averaging the metric as calculated in accordance with any of the embodiments described above over a second time window. For example, if DPOP is used as the measure of fluid responsiveness, and is calculated over a fixed duration of 10 seconds, it may be desirable to average the plurality of DPOP calculations performed over a fixed window of 120 seconds, effectively taking the average of 12 DPOP calculations to yield a parameter indicative of the subject's fluid responsiveness.

Because fluid responsiveness has been shown to correlate with respiratory variations, it is desirable to isolate respiratory-induced modulations by removing all non-respiratory components of the PPG signal, and determine fluid responsiveness based only on the respiratory-induced modulations. In accordance with some embodiments of the present disclosure, a respiration rate of the subject is used to filter non-respiratory components of the PPG signal, and fluid responsiveness is determined based on the filtered signal.

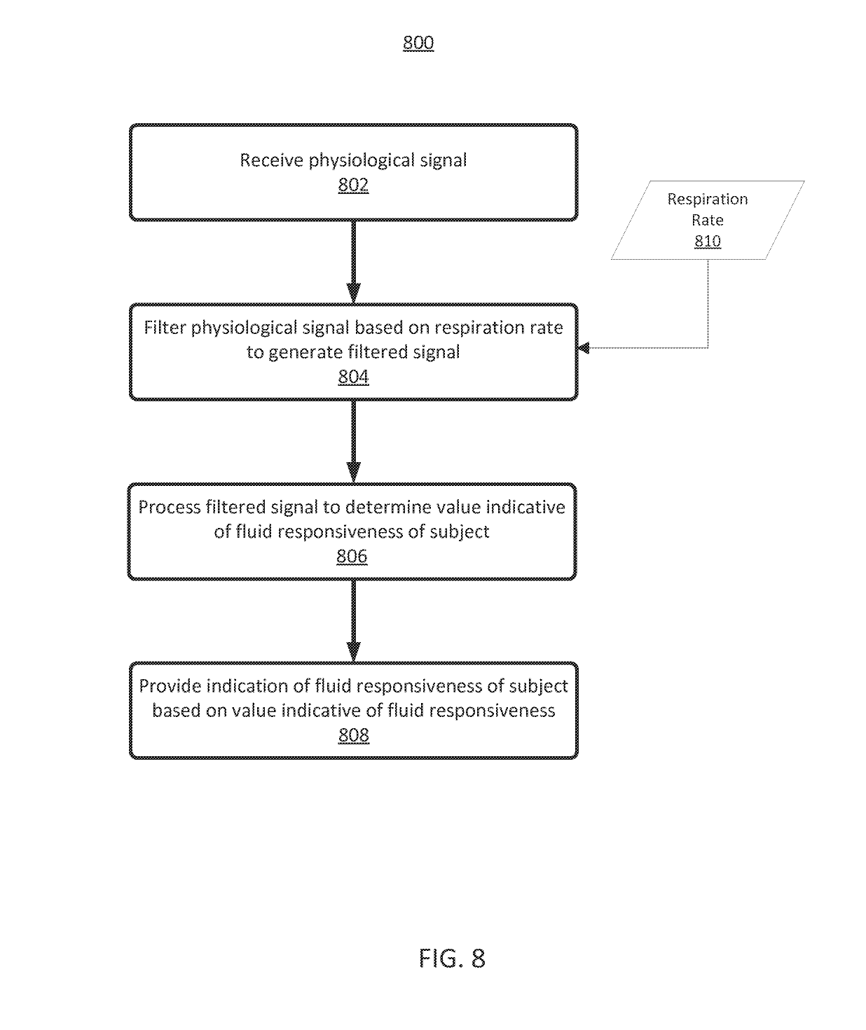

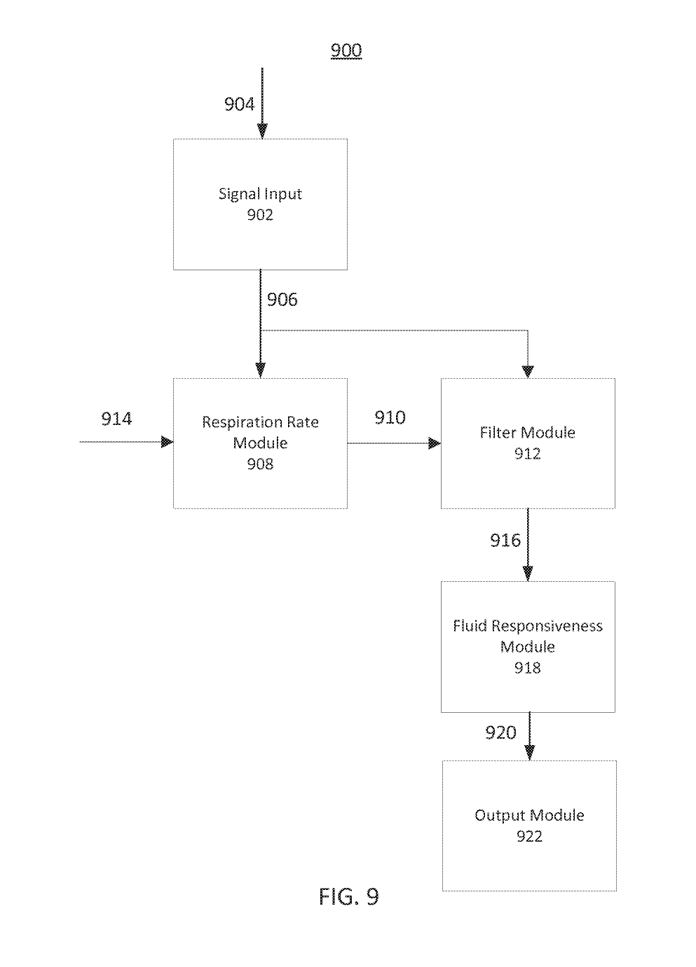

Filtering the PPG signal based on a subject's respiration rate and determining the subject's fluid responsiveness based on the filtered signal in accordance with the present disclosure will be discussed with reference to FIGS. 8-9 below.

FIG. 8 shows illustrative steps 800 for determining fluid responsiveness in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310, monitoring system 100, any components thereof, and any combination thereof.

At step 802, the physiological monitoring system may receive a physiological signal. In some embodiments, the physiological signal may be indicative of light attenuated by a subject. For example, the physiological signal may be a PPG signal received from a pulse oximeter as described above with respect to FIGS. 1-3. In some embodiments, the physiological signal may be a pressure signal. For example, the signal may be an arterial blood pressure signal. In some embodiments, the physiological signal may be a stroke volume signal. In some embodiments, the physiological signal may be any signal obtained from a subject and used in the determination of fluid responsiveness of the subject.

At step 804, the physiological monitoring system may filter the physiological signal based on a respiration rate 810 of the subject to generate a filtered signal. In some embodiments respiration rate 810 may be determined by the system based on the physiological signal received in step 802 in accordance with any of the methods described above to determine respiration rate. For example, the system may generate one or more morphology metrics from a PPG signal as described above with respect to FIGS. 4-6, and determine respiration rate based on the one or more morphology metrics. As described above the system may generate a series of morphology metric values and process the metric values to generate respiration morphology signals. The system may generate an autocorrelation sequence for each of the respiration morphology signals and identify peaks in the autocorrelation in order to determine periodic aspects of the underlying morphology signals. The system may calculate the respiration rate based on the periodic aspects of the underlying morphology signals. In some embodiments, respiration rate 810 may be calculated from any other physiological signal in any suitable manner. For example, the respiration rate may be calculated based on a PPG signal, a blood pressure signal, a stroke volume signal, a blood flow velocity signal, a capnography signal, a pressure pad signal, an accelerometer signal, a transthoracic impedance signal, a pneumotachometer signal, a nasal cannula signal, a microphone signal, a ventilator signal, a continuous positive airway pressure device signal, a bi-level positive airway pressure device signal, any other suitable physiological signal, and/or any suitable combination thereof. In some embodiments respiration rate 810 may be received from an external device via communication interface 190 or any other suitable interface. For example, the respiration rate may be received from a ventilator coupled to the physiological monitoring system via communication interface 190. In some embodiments, the external device may calculate respiration rate in any suitable manner using any suitable physiological signal described above, any other suitable physiological signals, and/or any suitable combination thereof.

In some embodiments, the system may apply a band-pass filter on the physiological signal to remove the non-respiratory-induced modulations including noise and other disturbances from the physiological signal based on the respiration rate of the subject. For example, the system may set the lower threshold of the pass band above the respiration rate, and the higher threshold of the pass band at a value which removes higher frequency noise in the signal. In some embodiments, the system may filter the signal based on the pulse rate of the subject.

At step 806, the physiological system may process the filtered signal to determine a value indicative of fluid responsiveness of the subject. In some embodiments, the filtered signal may be processed to identify morphology metrics, respiration morphology signals, respiratory modulations, and any combination thereof as described above with respect to FIGS. 4-7. For example, the filtered signal may be processed to determine respiratory modulations such as amplitude modulations, baseline modulations, and/or frequency modulations caused by respiration in the subject. A value indicative of fluid responsiveness may be determined based on the respiratory modulations. For example, the system may determine DPOP as described above by determining a maximum amplitude of the filtered signal over a time period, determining a minimum amplitude of the filtered signal over the same time period, and calculating DPOP based on the maximum and minimum amplitudes and Eqs. 1 and 2 above. In some embodiments, the value indicative of fluid responsiveness may be determined repeatedly over a time period corresponding to a breathing period of the subject. For example, DPOP may be determined repeatedly every 5 seconds, every 10 seconds, or at any other suitable interval. In some embodiments, the time period may be fixed, e.g. 5 or 10 seconds, or may be dynamically adjusted based on the respiration rate of the subject. In some embodiments, the system may take an average of the value indicative of fluid responsiveness over time. For example, the system may calculate DPOP repeatedly every 10 seconds, and take an average of each DPOP value calculated over another fixed time period, such as 120 seconds. In other words, the system may average 12 calculations of DPOP. In some embodiments, the system may repeat the averaging step over time.

At step 808, the system may provide an indication of the fluid responsiveness of the subject based on the value indicative of fluid responsiveness. In some embodiments, the system may output the value indicative of fluid responsiveness determined at step 806 continuously. In some embodiments, the system may output the value indicative of fluid responsiveness calculated repeatedly in step 806. In some embodiments, the system may output the average of the value indicative fluid responsiveness calculated repeatedly in step 806. In some embodiments, the system may provide the indication of fluid responsiveness on a display for use in diagnosis of a subject. For example, the indication of fluid responsiveness may be output to be displayed on display 320, display 328, display 184, or may be output to another device via communication interface 190, so that a clinician may diagnose a subject's condition and provide treatment in response thereto.

An illustrative physiological monitoring system 900 for monitoring fluid responsiveness of a subject is shown in FIG. 9. System 900 includes a signal input 902. In some embodiments, signal input 902 may include any suitable combination of components of monitor 100 for receiving a signal as described with respect to FIG. 1. For example, input 902 may include sensor 102, light drive circuitry 120, control circuitry 110, and front end processing circuitry 150 as described above with respect to FIG. 1, and may be configured to receive, generate and process signals as described above. In some embodiments, signal input 902 may include fewer components or additional components. Signal input 902 receives a physiological signal 904. In some embodiments, physiological signal 904 may be a signal indicative of light attenuated by a subject. For example, the physiological signal may be a PPG signal generated by a pulse oximeter as described above with respect to FIGS. 1-3. In some embodiments, physiological signal 904 may be any of the physiological signals described above with respect to step 802 of FIG. 8, such as a pressure signal, a stroke volume signal, or any other suitable physiological signal used to determine fluid responsiveness.

Signal input 902 generates output 906. Output 906 may include physiological signal 904, components thereof, processed versions thereof, or any suitable combination thereof. In some embodiments, output 906 is passed to respiration rate module 908. Respiration rate module 908 is coupled to signal input 902 and may be configured to determine respiration rate as described above with respect to steps 800. For example, respiration rate may be determined by generating a series of morphology metric values, generating respiration morphology signals based thereon, generating an autocorrelation sequence for each of the respiration morphology signals, identifying peaks in the autocorrelation in order to determine periodic aspects of the underlying morphology signals, and calculating the respiration rate based on the periodic aspects of the underlying morphology signals. In some embodiments, respiration rate module 908 may include any suitable combination of components of monitor 100 as described with respect to FIG. 1 for analyzing and processing a physiological signal. For example, respiration rate module 908 may include front end processing circuitry 150, back end processing circuitry 170, any components thereof, and/or any suitable combination thereof as described above with respect to FIG. 1, and may be configured to receive signals and process them as described above. In some embodiments, respiration rate module 908 may include fewer components or additional components. Respiration rate module 908 generates output 910 that is passed to filter module 912. Output 910 may include the respiration rate. In some embodiments, output 906 may also be passed to filter module 912 by signal input 902.

In some embodiments, respiration rate module 908 may be replaced by a respiration rate input that receives a respiration rate value 914 for a subject and passes the respiration rate value 914 to filter module 912. Respiration rate input may include communication interface 190 configured to receive a respiration rate calculated by an external device. In some embodiments respiration rate input may be used in conjunction with respiration rate module to provide for multiple calculations of respiration rate of the subject from which a suitable rate is chosen. For example, respiration rate module 908 may receive a respiration rate value 914 calculated by an external device such as a ventilator and may calculate its own respiration rate, and determine which to pass to filter module 912 based on any suitable criteria such as confidence metrics associated with the calculations or underlying signals.

In some embodiments, filter module 912 is configured to filter the physiological signal based on outputs 906, 910, and/or respiration rate value 914 to generate a filtered signal. In some embodiments, filter module 912 may filter the physiological signal as described above with respect to step 804 of FIG. 8. For example, filter module 912 may filter physiological signal included in output 906 by applying a band-pass filter on the physiological signal with a lower threshold set to be above the respiration rate included in output 910 and/or respiration rate value 914. In some embodiments, filter module 912 may set a higher threshold of the pass band at a value which removes higher frequency noise in the physiological signal. In some embodiments, the filter module 912 may filter the physiological signal based on the pulse rate of the subject, as determined by monitoring systems 100 or 310, or any components thereof. In some embodiments, filter module 912 may include any suitable combination of components of monitor 100 as described with respect to FIG. 1 for analyzing and processing a physiological signal. For example, filter module 912 may include front end processing circuitry 150, back end processing circuitry 170, any components thereof, and/or any suitable combination thereof as described above with respect to FIG. 1, and may be configured to receive signals and process them as described above. In some embodiments, filter module 912 may include fewer components or additional components. Filter module 912 generates output 916 that is passed to fluid responsiveness module 918. Output 916 may include the filtered signal.

In some embodiments, fluid responsiveness module 918 may determine a value indicative of fluid responsiveness 920 in accordance with any of the above-mentioned techniques, including those discussed above with respect to FIGS. 7 and 8, and pass it to output module 922. For example, fluid responsiveness module 918 may determine a value indicative of fluid responsiveness repeatedly over 10 second intervals based on the filtered signal included in output 916, and determine an average of the values indicative of fluid responsiveness repeatedly over 120 second intervals and pass one or both of these values to output module 922. In some embodiments, fluid responsiveness parameter determination module 918 may include any suitable combination of components of monitor 100 as described with respect to FIG. 1 for analyzing and processing a physiological signal. For example, fluid responsiveness parameter determination module 918 may include front end processing circuitry 150, back end processing circuitry 170, any components thereof, and/or any suitable combination thereof as described above with respect to FIG. 1, and may be configured to receive signals and process them as described above. In some embodiments, fluid responsiveness parameter determination module 918 may include fewer components or additional components.

Output module 922 may include display 184 and/or communication interface 190 of monitor 104 as described above with respect to FIG. 1, displays 320 and/or 328 of physiological monitoring system 310 as described above with respect to FIG. 3, any other suitable output, or any other suitable combination thereof. For example, the value indicative of fluid responsiveness or the average thereof may be output to be displayed on display 320, display 328, display 184, or may be output to another device via communication interface 190, so that a clinician may diagnose a subject's condition and provide treatment in response thereto.

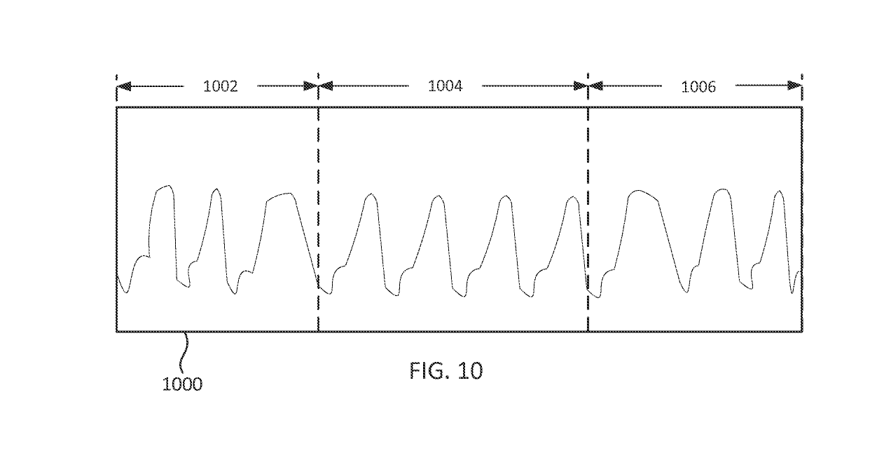

As described above, variability in fluid responsiveness determinations also arises from the manner of breathing exhibited by the subject. Specifically, studies have shown that the correlation between DPOP and PPV is particularly strong when DPOP is determined during periods of controlled and/or regular breathing by the subject, as opposed to periods of irregular or sporadic breathing, where the correlation between DPOP and PPV can be degraded. Further embodiments in accordance with the present disclosure for determining fluid responsiveness during regular breathing and/or controlled breathing will be discussed with reference to FIGS. 10-12 below.

In accordance with some embodiments of the present disclosure, regular breathing in the subject may be detected, and fluid responsiveness may be determined based primarily on periods of regular breathing. FIG. 10 shows an illustrative plot 1000 of respiratory flow with periods of regular and irregular breathing in accordance with some embodiments of the present disclosure. Because it has been found that the correlation between DPOP or other fluid responsiveness parameters and PPV, and in turn, the accuracy of DPOP or other fluid responsiveness parameters, may be degraded during times of irregular breathing, and may be strengthened during times of regular breathing, it may be desirable to detect periods of irregular and/or regular breathing in accordance with embodiments of the present disclosure. As can be seen in FIG. 10, a subject may experience periods with irregular breathing patterns, such as periods 1002 and 1006, which may result in certain characteristics indicative of irregular breathing patterns, such as the periods, amplitudes, morphologies, or other characteristics in any number of physiological signals obtained from the subject, including, e.g., in a PPG signal of the subject. Similarly, a subject may experience periods with regular breathing patterns, such as period 1004, which may result in certain characteristics indicative of a regular breathing pattern, such as the period, amplitude, morphology, or other characteristics in any number of physiological signals obtained from the subject, including, e.g., in a PPG signal of the subject. In view of the foregoing, it may be desirable to detect irregular breathing periods 1002 and 1006 and/or regular breathing period 1004, and only determine or display fluid responsiveness during regular breathing period 1004 so as to obtain accurate fluid responsiveness values that correlate to PPV in the subject non-invasively.

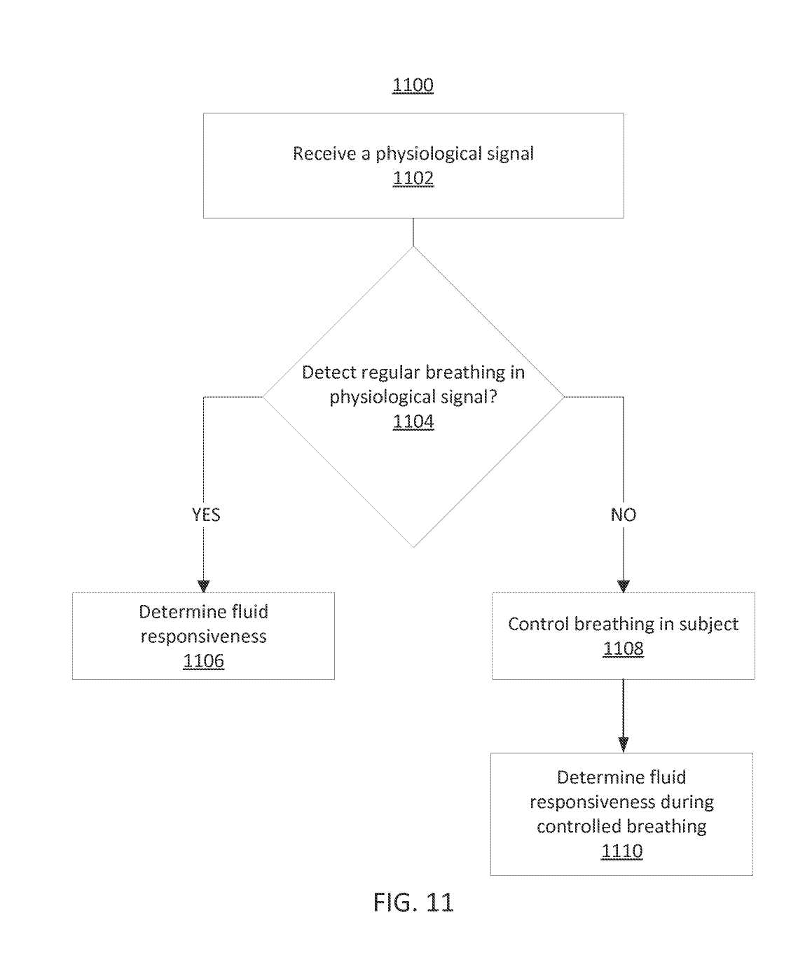

FIG. 11 shows illustrative steps 1100 for determining fluid responsiveness in accordance with some embodiments of the present disclosure. Although exemplary steps are described herein, it will be understood that steps may be omitted and that any suitable additional steps may be added for determining respiration information. Although the steps described herein may be performed by any suitable device or system, in an exemplary embodiment, the steps may be performed by monitoring system 310, monitoring system 100, any components thereof, and any combination thereof.

At step 1102, the physiological monitoring system may receive one or more physiological signals. In some embodiments, the physiological signal may be indicative of light attenuated by a subject. For example, the physiological signal may be a PPG signal received from a pulse oximeter as described above with respect to FIGS. 1-3. In some embodiments, the physiological signal may be a pressure signal. In some embodiments, the physiological signal may be a stroke volume signal. In some embodiments, the physiological signal may be any signal obtained from a subject and used in the determination of fluid responsiveness of the subject.

At step 1104, the physiological monitoring system may detect whether regular breathing is present based on, for example, analyzing the physiological signal. In some embodiments, the physiological monitoring system may generate a measurement of the regularity of the subject's breathing based on characteristics of the subject's breathing pattern as evidenced by the physiological signal. In some embodiments, the characteristics may include the respiratory pressure per breath, the breath period, the morphology of flow, the morphology of pressure, respiration rate, any other suitable characteristics of the subject's breathing pattern, or any suitable combination thereof. In some embodiments, the system may detect regular breathing based on other parameters, such as a period of stationary heart rate, blood pressure, oxygen saturation, fluid responsiveness, any other suitable parameter, and/or any suitable combination thereof. In some embodiments, any of the characteristics or parameters above may be analyzed over time to determine if the subject's breathing is regular. For example, the subject's respiration rate may be analyzed over time to determine if the subject's breathing is regular. For example, the respiration rate may be compared to a threshold, a measure of variability or consistency of the respiration rate may be compared to a threshold, or any other aspect of the respiration rate may be compared to a predetermined threshold to determine if the subject's breathing rate is regular.

If regular breathing is detected at step 1104, the system may proceed to step 1106, and it may determine fluid responsiveness during the regular breathing period in accordance with any of the techniques described in the present disclosure, including those described above with respect to FIGS. 7-9. For example, the system may determine maximum and minimum amplitudes within a time period of the detected regular breathing period and determine DPOP according to the maximum and minimum amplitudes and Eqs. 1 and 2 above.

In some embodiments, if regular breathing is not detected, the system will determine to refrain from determining fluid responsiveness. For example, if regular breathing is not detected, the system may cease calculating DPOP or any other fluid responsiveness measure. In some embodiments, if regular breathing is not detected, the system will refrain from outputting or otherwise displaying a calculated fluid responsiveness measure. For example, if regular breathing is not detected, the system will continue to calculate DPOP, but will not display the calculated DPOP when regular breathing is not detected.

In some embodiments, if regular breathing is not detected, the system may proceed to step 1108 and control breathing in the subject to obtain regular breathing. In some embodiments, the system may control breathing of the subject by sending a command to an external device to control the subject's breath. For example, the system may send a command via communication interface 190 to a ventilator attached to the subject to control the subject's breathing in any suitable manner to obtain regular breathing. In some embodiments, the breathing of the subject may be controlled by instructing the subject to breathe in a regular manner. For example, the system may output prompts to the subject via any of display 184, communication interface 190, display 320 and/or display 328 indicating when to breath, how much effort with which to breath, or both. In some embodiments, a clinician may instruct the subject how to breathe. In some embodiments, the breathing of the subject may be controlled by providing treatment to obtain regular breathing. For example, the subject may be sedated in order to obtain regular breathing. In some embodiments, the breathing of the subject may be controlled for several breaths. In some embodiments, the breathing of the subject may be controlled to generate a period of no breathing. For example, a ventilator may control the subject's breathing to generate a period of 10 seconds of no breathing. In another example, any of the above displays or a clinician may prompt or instruct a subject not to breath for 10 seconds.

At step 1110, if breathing is controlled according to step 1108, the system may then determine fluid responsiveness during the controlled breathing period. As described above with respect to step 1106, the system may determine fluid responsiveness in accordance with any of the techniques described in the present disclosure, including those described above with respect to FIGS. 7-9. For example, the system may determine maximum and minimum amplitudes within a time period of the controlled regular breathing period and determine DPOP according to the maximum and minimum amplitudes and Eqs. 1 and 2 above.

In some embodiments, breathing may be controlled as described above with respect to step 1108 irrespective of step 1104. For example, breathing may be controlled by the system without detecting whether current breathing is regular or irregular, and then fluid responsiveness may be determined as described above with respect to step 1110 during the controlled breathing period. In some embodiments, where the subject is utilizing a ventilator with varying degrees of controlled breathing, the system may detect the degree of control utilized by the ventilator and determine whether to determine fluid responsiveness based thereon. For example, if a ventilator is in a highly controlled breathing mode corresponding to periods where the ventilator provides active assistance to the subject, the system may determine fluid responsiveness. In some embodiments, where the system detects that the ventilator is not operating in a highly controlled mode, the system may direct the ventilator to change into a highly controlled mode, and then determine fluid responsiveness during this period of highly controlled breathing. In some embodiments, the system may respond to a manual request for a fluid responsiveness measure by performing steps 1108 and 1110 as described above. For example, a clinician may request a fluid responsiveness measure via user interface 180, and the system may respond by directing a ventilator via communication interface 190 to control the subject's breathing for a controlled breathing period, determining DPOP over the controlled breathing period, and then directing the ventilator to return to its previous settings.

In some embodiments, the measurement of regularity determined in step 1104 may be used as a quality or confidence metric for the resulting fluid responsiveness value, which may be output along with the fluid responsiveness value, or may be used in a determination as to whether to output the fluid responsiveness value. For example, if a relatively high measurement of regularity is determined in step 1104, the resulting fluid responsiveness value determined in step 1106 may be output. On the other hand, if a relatively low measurement of regularity is determined in step 1104, the fluid responsiveness value may not be determined, or may be determined, but not output. In some embodiments, the system may determine fluid responsiveness continuously or at specified regular intervals regardless of whether breathing is regular, as described above, and an indication of the accuracy of the fluid responsiveness value may be provided based on the measurement of regularity. For example, an indicator may be provided to the clinician that the fluid responsiveness value may not be accurate due to irregular breathing in the subject.

In some embodiments, the frequency and length of the controlled period of breathing as described above may be adaptive. In some embodiments, the frequency of controlled breathing may be a function of previous measurements (and their proximity to clinical decision points). For example, breathing may be controlled more or less often based on previous fluid responsiveness determinations, other physiological parameter determinations, or any other suitable measurements. In some embodiments, the length of the controlled period of breathing may be a function of the quality or confidence metric for the fluid responsiveness as described above. For example, the controlled period of breathing may continue until a desirable quality or confidence metric is obtained for the fluid responsiveness parameter.

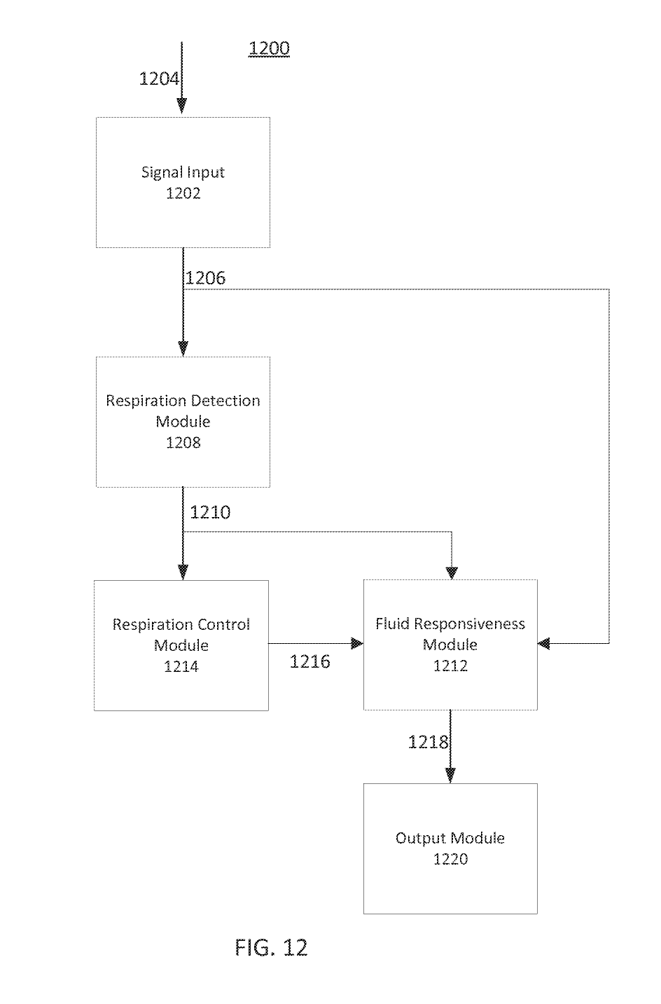

An illustrative physiological monitoring system 1200 for monitoring fluid responsiveness of a subject is shown in FIG. 12. System 1200 includes a signal input 1202. In some embodiments, signal input 1202 may include any suitable combination of components of monitor 100 for receiving a signal as described with respect to FIG. 1. For example, input 1202 may include sensor 102, light drive circuitry 120, control circuitry 110, and front end processing circuitry 150 as described above with respect to FIG. 1, and may be configured to receive, generate and process signals as described above. In some embodiments, signal input 1202 may include fewer components or additional components. Signal input 1202 receives a physiological signal 1204. In some embodiments, physiological signal 1204 may be a signal indicative of light attenuated by a subject. For example, the physiological signal may be a PPG signal generated by a pulse oximeter as described above with respect to FIGS. 1-3. In some embodiments, physiological signal 1204 may be any of the physiological signals described above with respect to step 1102 of FIG. 11, such as a PPG signal, a pressure signal, a stroke volume signal, or any other suitable physiological signal used to determine fluid responsiveness.

Signal input 1202 generates output 1206. Output 1206 may include physiological signal 1204, components thereof, processed versions thereof, or any suitable combination thereof. In some embodiments, output 1206 is passed to respiration detection module 1208. Respiration detection module 1208 is coupled to signal input 1202 and may be configured to detect periods of regular breathing as described above with reference to step 1104 of FIG. 11. For example, respiration detection module 1208 may generate a measurement of the regularity of the subject's breathing based on characteristics of the subject's breathing pattern including the respiratory pressure per breath, the breath period, the morphology of flow, the morphology of pressure, any other suitable characteristics of the subject's breathing pattern, or any suitable combination thereof. In some embodiments, respiration detection module 1208 may detect regular breathing based on other parameters, such as a period of stationary heart rate, blood pressure, oxygen saturation, fluid responsiveness, any other suitable parameter, and/or any suitable combination thereof. In some embodiments, respiration detection module 1208 may be coupled to a ventilator input (not shown) which may be configured to receive information from a ventilator being used by the subject. For example, the ventilator input may be coupled to an adjustable ventilator being used by the subject which has varying degrees of control over the subject's breathing. The ventilator input may receive an indication from the adjustable ventilator regarding the degree of control of the subject's breathing. In some embodiments, the ventilator input may generate an output based on the indication received from the adjustable ventilator and pass it to respiration detection module 1208. In some embodiments, the respiration detection module 1208 may detect regular and/or irregular breathing of a subject based on the output of the ventilator input. In some embodiments, respiration detection module 1208 may detect suppressed breathing of a subject. For example, respiration detection module 1208 may receive an indication from an external device such as a ventilator that the subject's breathing is being suppressed. In some embodiments, respiration detection module 1208 may include any suitable combination of components of monitor 100 as described with respect to FIG. 1 for analyzing and processing a physiological signal. For example, respiration detection module 1208 may include front end processing circuitry 150, back end processing circuitry 170, any components thereof, and/or any suitable combination thereof as described above with respect to FIG. 1, and may be configured to receive signals and process them as described above. In some embodiments, respiration detection module 1208 may include fewer components or additional components. Respiration detection module 1208 generates output 1210 that may be passed to fluid responsiveness module 1212 and respiration control module 1214. Output 1210 may include the measurement of regularity of the subject's breathing and/or an indication of regular or irregular breathing. In some embodiments output 1206 is also passed to fluid responsiveness module 1212.

In some embodiments, when an indication of regular breathing is received from the respiration detection module 1208, fluid responsiveness module 1212 may determine a value indicative of fluid responsiveness during the period of regular breathing in accordance with any of the above-mentioned techniques, including those discussed above with respect to FIGS. 7-9, and pass it to output module 1220. In some embodiments, when an indication of sufficiently controlled breathing is received from the ventilator input by the respiration detection module 1208, or an indication of suppressed breathing is received by the respiration detection module 1208, fluid responsiveness module 1212 may determine a value indicative of fluid responsiveness during the period of sufficiently controlled breathing or suppressed breathing in accordance with any of the above-mentioned techniques, including those discussed above with respect to FIGS. 7-9, and pass it to output module 1220. In some embodiments, fluid responsiveness module 1212 may determine a quality metric of the value indicative of fluid responsiveness based on the measurement of regularity included in output 1210, and pass it to output module 1220, or use it to determine whether to pass the value indicative of fluid responsiveness to output module 1220. For example, if there is a relatively high measurement of regularity, the value indicative of fluid responsiveness may be passed to output module 1220. On the other hand, if there is a relatively low measurement of regularity the value indicative of fluid responsiveness value may not be passed to output module 1220.

In some embodiments, fluid responsiveness parameter determination module 1212 may include any suitable combination of components of monitor 100 as described with respect to FIG. 1 for analyzing and processing a physiological signal. For example, fluid responsiveness parameter determination module 1212 may include front end processing circuitry 150, back end processing circuitry 170, any components thereof, and/or any suitable combination thereof as described above with respect to FIG. 1, and may be configured to receive signals and process them as described above. In some embodiments, fluid responsiveness parameter determination module 1212 may include fewer components or additional components.

In some embodiments, when an indication of regular breathing is not received from the respiration detection module 1208 or when an indication of irregular breathing is received from respiration detection module 1208, fluid responsiveness module 1212 may refrain from determining a value indicative of fluid responsiveness. In some embodiments, when an indication of regular breathing is not received from the respiration detection module 1208 or when an indication of irregular breathing is received from respiration detection module 1208, fluid responsiveness module 1212 may still determine a value indicative of fluid responsiveness, but may refrain from passing the value indicative of fluid responsiveness to output module 1220, and the value indicative of fluid responsiveness may not be displayed by output module 1220. In some embodiments, when an indication of regular breathing is not received from the respiration detection module 1208 or when an indication of irregular breathing is received from respiration detection module 1208, the value indicative of fluid responsiveness may be passed to output module 1220, but may not be displayed by output module 1220.

In some embodiments, when respiration detection module 1208 does not detect regular breathing, or detects irregular breathing, output 1210 may be passed to a respiration control module 1214. Respiration control module 1214 may be configured to control the breathing of the subject in accordance with the techniques described above with respect to step 1108 of FIG. 11. In some embodiments, respiration control module 1214 may control breathing of the subject by sending a command to an external device to control the subject's breath. For example, the respiration control module may include a ventilator adjustment output which may send a command to an adjustable ventilator attached to the subject to control the patient's breathing in any suitable manner to obtain regular breathing. For example, the ventilator adjustment output may generate a signal to cause the ventilator to increase its control of the subject's breathing. In some embodiments, respiration control module 1214 may control the subject's breathing by instructing the subject to breathe in a regular manner. For example, the respiration control module 1214 may output prompts to the subject to breathe at certain intervals. In some embodiments, the respiration control module 1214 may control the breathing of the subject by providing treatment to obtain regular breathing. For example, the subject may be sedated in order to obtain regular breathing.

In some embodiments, respiration control module 1214 may include any suitable combination of components of monitors 100 and 310 as described with respect to FIGS. 1 and 3 for analyzing and processing a physiological signal. For example, respiration control module 1214 may include front end processing circuitry 150, back end processing circuitry 170, communications interface 190, displays 320 and/or 328 any components thereof, and/or any suitable combination thereof as described above with respect to FIGS. 1 and 3, and may be configured to receive signals and process them as described above. In some embodiments, respiration control module 1214 may include a ventilator or any other suitable device for controlling breathing of a subject. In some embodiments, respiration control module may include fewer components or additional components.

Respiration control module 1214 may generate output 1216 and pass it to fluid responsiveness module 1212. Fluid responsiveness module 1212 may determine a value indicative of fluid responsiveness during the period of controlled breathing in accordance with any of the above-mentioned techniques, including those discussed above with respect to FIGS. 7-9, and pass it to output module 1220.

In some embodiments, respiration control module 1214 may provide information to a respiration control input (not shown) coupled to the fluid responsiveness module 1212. In some embodiments, respiration control module 1214 may be a device external to physiological monitoring system 1200 and may provide information to a respiration control input of physiological monitoring system 1200. For example, respiration control module 1214 may be an adjustable ventilator that provides varying degrees of control of breathing of the subject and provides an indication of the degree of control of breathing to the physiological monitoring system via respiration control input. Respiration control input may include any suitable combination of components of monitors 100 and 310 as described with respect to FIGS. 1 and 3 for receiving, analyzing and processing a signal. For example, respiration control input may include front end processing circuitry 150, back end processing circuitry 170, communications interface 190, displays 320 and/or 328 any components thereof, and/or any suitable combination thereof as described above with respect to FIGS. 1 and 3, and may be configured to receive signals and process them as described above.

Output module 1220 may include display 184 and/or communication interface 190 of monitor 104 as described above with respect to FIG. 1, displays 320 and/or 328 of physiological monitoring system 310 as described above with respect to FIG. 3, any other suitable output, or any other suitable combination thereof. For example, the value indicative of fluid responsiveness or an average thereof may be output to be displayed on display 320, display 328, display 184, or may be output to another device via communication interface 190, so that a clinician may diagnose a subject's condition and provide treatment in response thereto.

Although system 1200 has been described above with reference to both respiration detection module 1208 and respiration control module 1214, it will be understood that either of these modules may be optional, and in some embodiments, one of the modules may not be used as will be described below.

In some embodiments, respiration control module 1214 may be used without respiration detection module 1208, such that breathing of the subject is controlled irrespective of the regularity of the subject's breathing. For example, respiration control module 1214 may be controlled without detecting whether current breathing is regular or irregular, and then fluid responsiveness may be determined by fluid responsiveness module 1212 as described above during the controlled breathing period. In some embodiments, the respiration control module 1214 may respond to a manual request for a fluid responsiveness measure as described above. For example, a clinician may request a fluid responsiveness measure via user interface 180, and respiration control module 1214 may respond by directing a ventilator via communication interface 190 to control the subject's breathing for a controlled breathing period, and directing the fluid responsiveness module 1212 to determine DPOP over the controlled breathing period.

In some embodiments, respiration detection module 1208 may be used without respiration control module 1214, such that fluid responsiveness is only determined during periods of regular breathing detected by respiration detection module 1208.

It will be understood that while FIGS. 9 and 12 show separate systems for filtering a signal using respiration rate to generate a filtered signal from which to determine fluid responsiveness and detecting and controlling respiration in a subject to determine fluid responsiveness respectively, a system may be provided in accordance with the present disclosure that includes any combination of respiration rate module 908, filter module 912, respiration detection module 1208, and respiration control module 1214 so that fluid responsiveness can be more accurately determined.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A system, comprising:
a signal input that receives a plethysmograph signal;
a respiration rate module coupled to the input and configured to calculate a respiration rate of a subject based at least in part on the plethysmograph signal;
a filter module coupled to the input and to the respiration rate module, the filter module configured to filter the plethysmograph signal based at least in part on the respiration rate to generate a filtered signal;
a fluid responsiveness module coupled to the filter module and configured to process the filtered signal to determine a value indicative of fluid responsiveness of the subject; and
an output module configured to provide an indication of the fluid responsiveness of the subject based at least in part on the value indicative of fluid responsiveness.

2. The system of claim 1, wherein the respiration rate module is further configured to generate at least one morphology metric from the plethysmograph signal, and wherein the respiration rate is based at least in part on the at least one morphology metric.

3. The system of claim 2, wherein the at least one morphology metric is selected from the group consisting of a down metric, a kurtosis metric, a delta of the second derivative, an up metric, a skew metric, a b/a ratio metric, a c/a ratio metric, a i_b metric, a peak amplitude metric, a center of gravity metric, an area metric, and any combination thereof.

4. The system of claim 1, wherein the filter module comprises a band-pass filter having a pass band defined at least in part by the respiration rate.

5. The system of claim 4, wherein the pass band is further defined at least in part by a pulse rate of the subject.

6. The system of claim 1, wherein the filter module is further configured to remove components of the plethysmograph signal not related to pulses modulated by respiration.

7. The system of claim 1, wherein the fluid responsiveness module is further configured to analyze an amplitude modulation of the filtered signal caused by respiration.

8. The system of claim 1, wherein the fluid responsiveness module is further configured to determine the value indicative of fluid responsiveness repeatedly over a first time period by performing an operation comprising:
determining a maximum amplitude of the filtered signal over the first time period;
determining a minimum amplitude of the filtered signal over the first time period;
calculating a difference between the maximum amplitude and the minimum amplitude;
calculating an average of the maximum amplitude and the minimum amplitude;
calculating a ratio of the difference and the average; and
determining the value indicative of fluid responsiveness of the subject based on the ratio.

9. The system of claim 8, wherein the indication of the fluid responsiveness of the subject is based at least in part on an average of the value indicative of fluid responsiveness of the subject over time.

10. A system, comprising:
a respiration rate input that receives a respiration rate value for a subject;
a signal input that receives a plethysmograph signal;
a band-pass filter coupled to the respiration rate input and to the signal input, the band-pass filter configured to filter the plethysmograph signal to generate a filtered signal, wherein at least one characteristic of the band-pass filter is set based at least in part on the respiration rate;
a fluid responsiveness module coupled to the band-pass filter and configured to process the filtered signal to determine a value indicative of fluid responsiveness of the subject; and
an output module configured to provide an indication of the fluid responsiveness of the subject based at least in part on the value indicative of fluid responsiveness.

11. The system of claim 10, further comprising a respiration rate module coupled to the respiration rate input and configured to calculate the respiration rate based on at least one signal selected from the group consisting of a plethysmograph signal, a blood pressure signal, a stroke volume signal, a blood flow velocity signal, a capnography signal, a pressure pad signal, an accelerometer signal, a transthoracic impedance signal, a pneumotachometer signal, a nasal cannula signal, a microphone signal, a ventilator signal, a continuous positive airway pressure device signal, a bi-level positive airway pressure device signal, and any combination thereof.

12. The system of claim 10, band-pass filter is further configured to remove at least some modulations of pulses in the plethysmograph signal not caused by respiration.

13. The system of claim 10, further comprising a pulse oximetry module coupled to the signal input and that is configured to calculate a pulse rate based at least in part on the plethysmograph signal, wherein at least one other characteristic of the band-pass filter is set based on the pulse rate.

14. The system of claim 10, wherein the fluid responsiveness module is further configured to analyze an amplitude modulation of the filtered signal caused by respiration.

15. The system of claim 10, wherein the fluid responsiveness module is further configured to determine the value indicative of fluid responsiveness repeatedly over a first time period by performing an operation comprising:
determining a maximum amplitude of the filtered signal over the first time period;
determining a minimum amplitude of the filtered signal over the first time period;
calculating a difference between the maximum amplitude and the minimum amplitude;
calculating an average of the maximum amplitude and the minimum amplitude;
calculating a ratio of the difference and the average; and
determining the value indicative of fluid responsiveness of the subject based on the ratio.

16. The system of claim 15, wherein the indication of the fluid responsiveness of the subject is based at least in part on an average of the value indicative of fluid responsiveness of the subject over time.

17. A method, comprising:
receiving at a signal input a plethysmograph signal from a sensor attached to a subject;
filtering, using a filter module, the plethysmograph signal based on a respiration rate of the subject;
processing the filtered signal using a processor to determine a value indicative of fluid responsiveness of the subject; and
outputting on an output device an indication of the fluid responsiveness of the subject based at least in part on the value indicative of fluid responsiveness.

18. The method of claim 17, wherein the filtering the plethysmograph signal further comprises band-pass filtering the plethysmograph signal with a pass band defined at least in part by the respiration rate.

19. The method of claim 17, wherein the processing the filtered signal to determine the value indicative of fluid responsiveness of the subject is performed repeatedly over a first time period, and comprises:
determining a maximum amplitude of the filtered signal over the first time period;
determining a minimum amplitude of the filtered signal over the first time period;
calculating a difference between the maximum amplitude and the minimum amplitude;
calculating an average of the maximum amplitude and the minimum amplitude;
calculating a ratio of the difference and the average; and
determining the value indicative of fluid responsiveness of the subject based on the ratio.

20. The method of claim 19, wherein the indication of the fluid responsiveness is based at least in part on an average of the value indicative of fluid responsiveness of the subject over time.
